(19) United States
(12) Patent Application Publication
(10) Pub. No.: US 2022/0356210 A1
(43) Pub. Date: Nov. 10, 2022

(54) A SERIES OF INJECTABLE HYDROGELS SELF-ASSEMBLED FROM SHORT PEPTIDES FOR VARIOUS BIOMEDICAL APPLICATIONS

(71) Applicant: Agency for Science, Technology and Research, Singapore (SG)

(72) Inventors: Yiyan YANG, Singapore (SG); Shaoqiong LIU, Singapore (SG); Pang Kern Jeremy TAN, Singapore (SG); Zhan Yuin ONG, Singapore (SG)

(73) Assignee: Agency for Science, Technology and Research, Singapore (SG)

(21) Appl. No.: 17/629,338

(22) PCT Filed: Jul. 22, 2020
(86) PCT No.: PCT/SG2020/050424

§ 371 Date: Jan. 21, 2022

(30) Foreign Application Priority Data
Jul. 22, 2019 (SG) 10201906759W

(51) Int. Cl. C07K 7/08; C07K 7/06; A61P 31/00

(52) U.S. Cl. CPC C07K 7/08; C07K 7/06; A61P 31/00; A61K 38/00

(57) ABSTRACT

Described herein are peptides that may self-assemble into hydrogels. The peptide comprises an amino acid sequence of alternating hydrophobic amino acids (X) and hydrophilic amino acids (Y), wherein each hydrophobic amino acid is independently selected from isoleucine (I), valine (V) and leucine (L), each hydrophilic amino acid is independently selected from arginine (R), lysine (K), glutamic acid (E), and aspartic acid (D), at least one hydrophilic amino acid is selected from arginine and lysine, at least one hydrophilic amino acid is selected from glutamic acid and aspartic acid, and the amino acid sequence contains at least 8 amino acids. Further described is a composition comprising a hydrogel formed of the peptides in a beta-sheet conformation and water or a dried form of the hydrogel. The hydrogel may be used for growing cells. In another aspect, a hybrid hydrogel prepared from IIK12 (IRIKIEIEIRIK) and IK8L (IRIKIRIK) may be used to treat a bacterial and/or fungal infection.

### TECHNICAL FIELD

The present invention relates to peptides that self-assemble into hydrogels.

### INTRODUCTION

Hydrogels, 3D polymeric networks capable of storing large amounts of water, have been extensively studied and used as matrix for biomedical applications[1]. Particularly, they are commonly chosen for regenerative medicine applications because of their biocompatibility, innate structural similarity to the extracellular matrix (ECM), and ability to provide suitable biochemical environments[2, 3].

There have been many types of hydrogels reported in the literature, where both natural and synthetic materials are employed. Matrigel[4, 5].collagen[6, 7], hyaluronic acid [8] and gelatin[9] have been widely used to form hydrogels-based scaffolds for tissue engineering because of their advantage of being specifically recognized by host cells. However, the use of the animal derived scaffolds is often restricted because of the undefined or inconsistent molecule length and structure as well as potential risk of immunogenicity[10]. Polymers based on poly(ethylene glycol)[10, 11], poly(vinyl alcohol)[12], poly(L-lactide) (PLLA)[13], poly(lactide-co-glycolide) (PLGA)[14] and poly(hydroxyethyl methacrylate) (HEMA)[15] have also been widely used to create hydrogels. However, synthetic polymeric hydrogels is biochemically inert in nature and unable to interact with cells, and thus it is difficult to allow the cells to proliferate within the matrix[10].

Recently, much attention has been given to peptide hydrogels. Peptide-based hydrogels derived from naturally occurring amino acids offer several advantages such as biocompatibility, biodegradability and non-immunogenicity[16, 17]. Production cost has also been greatly reduced with the advances in solid phase peptide synthesis. Moreover, each amino acid coupling step is precisely controlled so that well-defined sequence, molecular length and reproducibility are easily achievable. In the early 1990s, Zhang and co-workers discovered a natural yeast protein motif, EAK16-HI (AEAEAKAKAEAEAKAK)(SEQ ID No. 1), characterized by alternative repeats of ionic hydrophilic and hydrophobic amino acids[17]. This peptide adopted a β-sheet configuration with distinct hydrophobic and hydrophilic surfaces, resulting in self-assembled nanofibers in the presence of salt. Since then, a series of peptide self-assembly systems have been designed and developed to form 3D hydrogels with nanofiber structure. The most extensively studied peptides RADA16-I (AcN-RADARADARADARADA-CONH₂) (SEQ ID No. 2) [16](PuraMatrix™) and RADA-II (AcN-RARADADARARADADA-CONH₂) (SEQ ID No. 3)[18] consist of periodic alternating hydrophilic arginine residue and hydrophobic alanine (A) residue. The peptide scaffolds self-assembled from RADA16-I and RADA16-II formed hydrogels (99% water) with nanofibers (10-20 nm in diameter) in physiological solutions. These peptide hydrogels were used as scaffolds for 3D cell culture, accelerated wound healing and nerve repair[19-21]. For instance, the scaffolds can support neurite outgrowth and synapse formation[21]. In addition, the RADA16 hydrogels have been utilised for delivery of proteins and active cytokines in a sustained release manner[22].

Schneider, Pochan and co-works developed another class of peptides with a central tetrapeptide type II′ β-turn (V^{D}PPT) flanked by alternating valine (hydrophobic) and lysine (hydrophilic) residues[23, 24]. Upon triggered by pH, ionic and temperature changes, peptides such as MAXI (VKVKVKVK-V^{D}PPT-VKVKVKVK) (SEQ ID No. 4) transformed from random coil to β-hairpin conformations and self-assembled into hydrogels rich in β sheet. MAXI was shown to be non-hemolytic towards human red blood cells and support the attachment of NIH3T3 cells[24, 25]. However, MAXI hydrogel was not ideal for 3D cell encapsulation due to its slow gelation kinetics[26].

Recently, Hauser et al. reported a new series of ultrashort linear peptides with 3-7 natural aliphatic amino acids that self-assemble into helical fibres in supramolecular structures[27, 28]. The peptide motif contains an aliphatic amino acid tail with decreasing hydrophobicity and a hydrophilic polar head. The peptides underwent secondary conformational transitions from random coil to α-helices to β sheets as the concentration increased to their critical gelation concentration. The peptide self-assembled into fibers via α-helical intermediate and subsequently condensed into fibrils to form a hydrogel[27]. The resulting peptides entrapped up to 99.9% water and resembled collagen fibers. The mechanical stiffness ranged from 10³ to 10⁵ Pa, which could be tuned by the peptide concentration and use of salts[28]. The peptides demonstrated biocompatibility to human mesenchymal stem cells (hMSCs) and rabbit retinal epithelial cells[28]. The lead peptide, Ac-ILVAGK-NH₂(SEQ ID No. 5), was evaluated for wound healing application[29]. The peptide hydrogel promoted the healing of partial-thickness burn wounds as compared to commercial wound dressing Mepitel®. Nonetheless, when soaked in a large volume of water, the fibers were diluted, causing the peptide hydrogel to lose its integrity[30].

More recently, hydrogels were reported for use in combating infections associated with medical device and wound healing. Diabetic foot ulcer (DFU) infections are the major cause of morbidity, hospitalization and the leading cause of amputations[31]. Chronic CFU is extremely difficult to treat as it involves a diversity of bacteria (including Gram-positive and Gram-negative bacteria, especially multidrug-resistant ones) and biofilm[32]. Most existing antibiotics are ineffective in treating such infections. Silver ion or silver nanoparticles have been incorporated into wound dressings for prevention of DFU infection[33, 34]. Long-term use of such products causes silver accumulation in skin, resulting in blue or grey discoloration condition of the skin (argyria). Therefore, biodegradable antimicrobial hydrogels with broad-spectrum activity against multidrug-resistant microbes, and capable of dispersing biofilms without developing drug resistance are urgently needed for prevention and treatment of DFU infection.

### SUMMARY

In a first aspect of the invention, there is provided a peptide comprising an amino acid sequence of alternating hydrophobic amino acids (X) and hydrophilic amino acids (Y), wherein each hydrophobic amino acid is independently selected from isoleucine (I), valine (V) and leucine (L), each hydrophilic amino acid is independently selected from arginine (R), lysine (K), glutamic acid (E), and aspartic acid (D), at least one hydrophilic amino acid is selected from arginine and lysine, at least one hydrophilic amino acid is selected from glutamic acid and aspartic acid, and the amino acid sequence contains at least 8 amino acids.

Amino acids typically refer to the L-amino acids, however it is possible for the D-amino acids to be used as well. In particular, the peptides described herein may be made of all L-amino acids or all D-amino acids. Peptide sequences described herein include both the all L-amino acid sequence and the all D-amino acid enantiomer unless stated otherwise, which are known to have identical properties except for the ability to rotate plane polarised light.

The peptides also include two or more of the amino acid sequences joined by a linker. Such peptides are also likely to form beta-sheets in water and self-assemble into hydrogel at lower peptide concentration. The linker may be any conventional organic compound linker and may be joined to the respective C-terminus and N-terminus.

Preferably, the amino acid sequence is not IRVEIEVK.

Preferably, the amino acid sequence has an even number of amino acids. Advantageously, this allows the peptide to maintain chemical complementarity, in other words equal number of hydrophilic and hydrophobic amino acid residues. For example, the amino acid sequence may have 8 or 12 amino acids. The latter is believed to form hydrogels more easily at lower concentrations.

Preferably, there is at least one arginine and at least one lysine in the amino acid sequence.

In an example, four sequential hydrophilic amino acids (Y₁, Y₂, Y₃ and Y₄) in the amino acid sequence of alternating hydrophobic amino acids (X) and hydrophilic amino acids (Y) are selected such that Y₁ and Y₂ are each independently selected from glutamic acid and aspartic acid, and Y₃ and Y₄ are each independently selected from arginine and lysine. Advantageously, this provides a peptide with a (− − + +) arrangement of the hydrophilic amino acid residues.

The phrase “sequential hydrophilic amino acids” as used herein refers to the consecutive hydrophilic amino acids in the alternating sequence of hydrophilic amino acids (Y) and hydrophobic amino acids (X). For example, four sequential (hydrophilic) amino acids in the peptide mean the peptide contains a sequence of Y₁X₁Y₂X₂Y₃X₃Y₄. A peptide with 8 amino acids would have the sequence Y₁X₁Y₂X₂Y₃X₃Y₄X₄, while the sequence is contained in peptides with 8 or more amino acids. The terms Y₁, Y₂, Y₃ and Y₄ denote the sequential order of the hydrophilic amino acids. The same applies to any number of sequential amino acids.

At a pH of about 7 (or 7.4) or under physiological conditions, arginine and lysine are positively charged while glutamic acid and aspartic acid are negatively charged. The selection of the hydrophilic amino acids thus provides for a peptide with localised charged characteristics that allows for the tuning of the properties of the peptide and may improve the resultant properties of the hydrogel formed from the peptide.

In an example, four sequential hydrophilic amino acids (Y₁, Y₂, Y₃ and Y₄) in the amino acid sequence of alternating hydrophobic amino acids (X) and hydrophilic amino acids (Y) are selected such that Y₁ and Y₃ are each independently selected from arginine and lysine, and Y₂ and Y₄ are each independently selected from glutamic acid and aspartic acid. Advantageously, this provides a peptide with a (+ − + −) arrangement of the hydrophilic amino acid residues.

In an example, six sequential hydrophilic amino acids (Y₁, Y₂, Y₃, Y₄, Y₅, and Y₆) in the amino acid sequence of alternating hydrophobic amino acids (X) and hydrophilic amino acids (Y) are selected such that Y₁, Y₃, and Y₅ are each independently selected from arginine and lysine, and Y₂, Y₄, and Y₆ are each independently selected from glutamic acid and aspartic acid. Advantageously, this provides a peptide with a (+ − + − +−) arrangement of the hydrophilic amino acid residues and may be viewed as the extension of the example with four sequential amino acids with the same alternating charged species arrangement.

In an example, six sequential hydrophilic amino acids (Y₁, Y₂, Y₃, Y₄, Y₅, and Y₆) in the amino acid sequence of alternating hydrophobic amino acids (X) and hydrophilic amino acids (Y) are selected such that Y₁, Y₂, and Y₃ are each independently selected from glutamic acid and aspartic acid, and Y₄, Y₅, and Y₆ are each independently selected from arginine and lysine. Advantageously, this provides a peptide with a (− − − + ++) arrangement.

In an example, six sequential hydrophilic amino acids (Y₁, Y₂, Y₃, Y₄, Y₅, and Y₆) in the amino acid sequence of alternating hydrophobic amino acids (X) and hydrophilic amino acids (Y) are selected such that Y₁, Y₂, Y₅, and Y₆ are each independently selected from arginine and lysine, and Y₃, and Y₄ are each independently selected from glutamic acid and aspartic acid. Advantageously, this provides a peptide with a (+ + − − + +) arrangement. In addition, the reverse sequence of hydrophilic amino acid residues in the examples above are also described and included herein.

Preferably, the hydrophilic amino acids are selected such that the peptide has a net neutral charge or net positive charge. For example, the peptide may have a net positive charge of +2. Advantageously, peptides with a net positive charge may form the hydrogel more readily without needing a salt to decrease the gelation time and at lower concentration thus leading to lower costs in the preparation of the hydrogels. This may be due to the enhanced beta sheet hydrogen bonds in peptides with a net positive charge.

Preferably, at least half of the hydrophobic amino acids in the amino acid sequence are isoleucine or leucine.

Preferably, there are 12 amino acids in the amino acid sequence and the hydrophobic amino acid residue is each independently selected from isoleucine and valine.

In an example, the amino acid sequence is any one of the following: SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 19, SEQ ID No. 20, SEQ ID No. 21, SEQ ID No. 22, SEQ ID No. 23, SEQ ID No. 24, SEQ ID No. 25, and SEQ ID No. 26. Further, the peptide may be made up of 2 or more of these sequences joined by a linker, in particular the sequences are of the same SEQ ID No.

Preferably, the peptide is amidated at a C-terminus of the amino acid sequence and/or acetylated at a N-terminus of the amino acid sequence. The C-terminus is preferably amidated in all peptides. A hydrophilic amino acid residue at the N-terminus may be acetylated, whereas a hydrophobic amino acid at the N-terminus may not require functionalisation of the N-terminus. Other functionalisation of the peptide ends may also be employed. Advantageously, this minimises charge repulsion at the terminus of the peptide.

In a second aspect of the invention, there is provided a composition comprising a hydrogel formed of the plurality of peptides according to the first aspect in a beta-sheet conformation and water or a dried form of the hydrogel. Advantageously, the formation of the beta-sheets form a supramolecular structure with rigid viscoelastic properties that is able to act as a hydrogel which can contain water and other compounds, and may be useful in different applications.

The dried form of the hydrogel may be obtained by freeze drying to increase the stability and storage time, and may be reconstituted back to the hydrogel by the addition of water. If the dried hydrogel contains a therapeutic agent, for example a second peptide of SEQ ID No. 17 or SEQ ID No. 18, the dried hydrogel may be reconstituted by at the point of use, for example the patient or medical professional.

Preferably, a concentration of the plurality of peptides is at least 0.6% w/v. The remainder of the hydrogel would be water, and forms the bulk of the hydrogel.

Preferably, the concentration is at most 10% w/v, at most 5% w/v. More preferably, the concentration is at most 4% w/v, or less than 4% w/v, or at most 3% w/v.

Preferably, the composition further comprises a salt. The salt may be phosphate buffered saline (PBS), Dulbecco's modified Eagle's medium (DMEM), or minimal essential medium (MEM). Salts like sodium or potassium salts may also be used, and are preferably neutral or weakly acidic or basic salts for example sodium chloride and sodium acetate.

In an example, the peptide has the amino sequence of SEQ ID No. 12, SEQ ID No. 15, SEQ ID No. 16, SEQ ID No. 22, SEQ ID No. 25 or SEQ ID No. 26.

Preferably, the composition or hydrogel comprises a therapeutic agent. More preferably, the composition or hydrogel comprises a second peptide of SEQ ID No. 17 or SEQ ID No. 18 (which acts as a therapeutic agent). The molar ratio of the peptide to the second peptide may be in the range of 3:1 to 1:3 (or alternatively 3 to 0.33). Advantageously, this composition or hydrogel may be used as a medicament. In particular, as a medicament for a bacterial infection and/or a fungal infection. Further, the composition or hydrogel may be used in the manufacture of a medicament for the treatment of a bacterial infection and/or a fungal infection. In an example, the fungal infection may be caused be a yeast.

In a third aspect, there is provided a method of treating a bacterial and/or a fungal infection, the method comprising administering a therapeutically effective amount of the composition or hydrogel containing a plurality of peptides according to the first aspect and a second peptide of SEQ ID No. 17 or SEQ ID No. 18 to a subject suffering from a bacterial and/or a fungal infection.

In a fourth aspect, there is provided a method to form a hydrogel, the method comprising mixing a peptide according to the first aspect in water to form a hydrogel; and isolating the hydrogel.

Preferably, the stirring step is done in the presence of a salt.

Preferably, the stirring step is done at a temperature of 20° C. to 40° C.

Preferably, the method further comprises drying the hydrogel to form a dried hydrogel suitable for reconstitution to the hydrogel.

Preferably, a concentration of the plurality of peptides is at least 0.6% w/v. The remainder of the hydrogel would be water which forms the bulk of the hydrogel.

Preferably, the concentration is at most 10% w/v, at most 5% w/v. More preferably, the concentration is at most 4% w/v, or less than 4% w/v, or at most 3% w/v, or at most 2% w/v,

Preferably, the composition further comprises a salt. The salt may be phosphate buffered saline (PBS), Dulbecco's modified Eagle's medium (DMEM), or minimal essential medium (MEM). Salts like sodium or potassium salts may also be used, and are preferably neutral or weakly acidic or basic salts, for example sodium chloride and sodium acetate.

In a fifth aspect, there is provided an in-vitro method of growing cells, the method comprises providing a mixture of the peptide according to the first aspect, cell culture medium, and a population of cells to form the gel containing the population of cells; and incubating the gel under suitable conditions to grow the population of cells.

Preferably, the cell is any one selected from a healthy cell, stem cell which is used for therapy or to grow tissues, and a cancer cell which is used to grow tumor for in vitro and in vivo studies. In an example, the stem cells may be human stem cells, and the formed hydrogel may be used as a medium to culture (or grow) the stem cells for use. The stem cells are preferably adult stem cells like human mesenchymal stem cells (hMSC). The hydrogel may also be used to grow cancer cells for in-vitro and/or in-vivo testing of therapeutic agents against cancer cells. Many cancer cell lines are available and may be used with the hydrogel to culture the cell line for further use, and include breast cancer cell lines like MCF-7 and BT-474.

In the Figures:

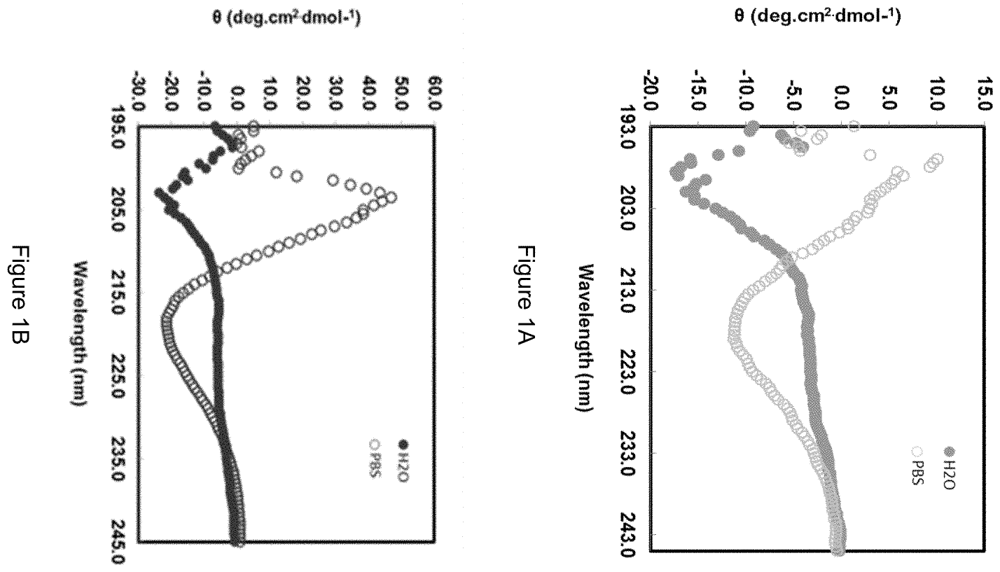

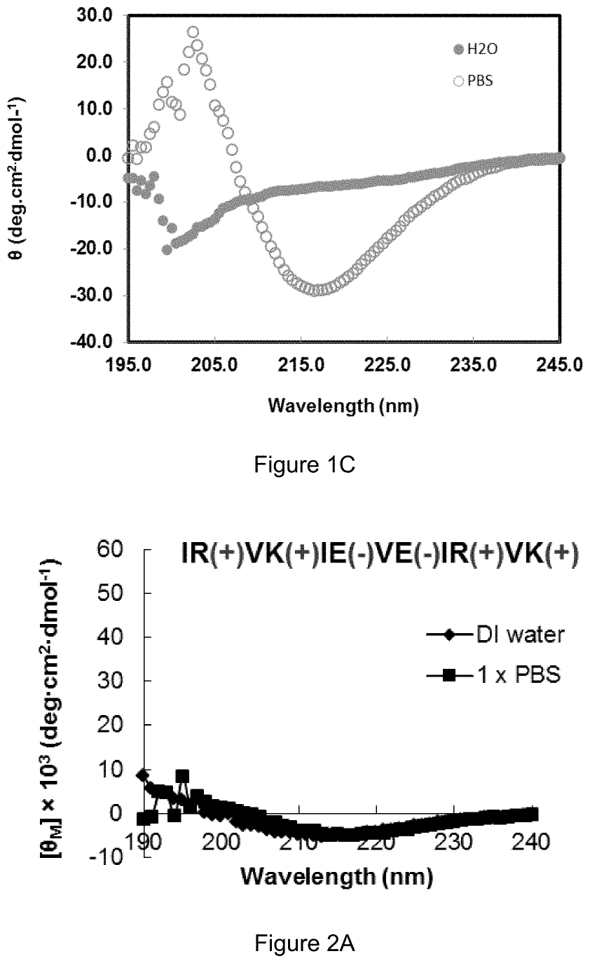

FIG. 1 shows the CD spectra of peptide amphiphiles consisting of 8 amino acids. FIG. 1A is for IVK8; FIG. 1B is for ILK8 and FIG. 1C is for IIK8. Measurements were carried out at 1.0 mg/mL in water or PBS (pH 7.4) at room temperature (˜23-24° C.).

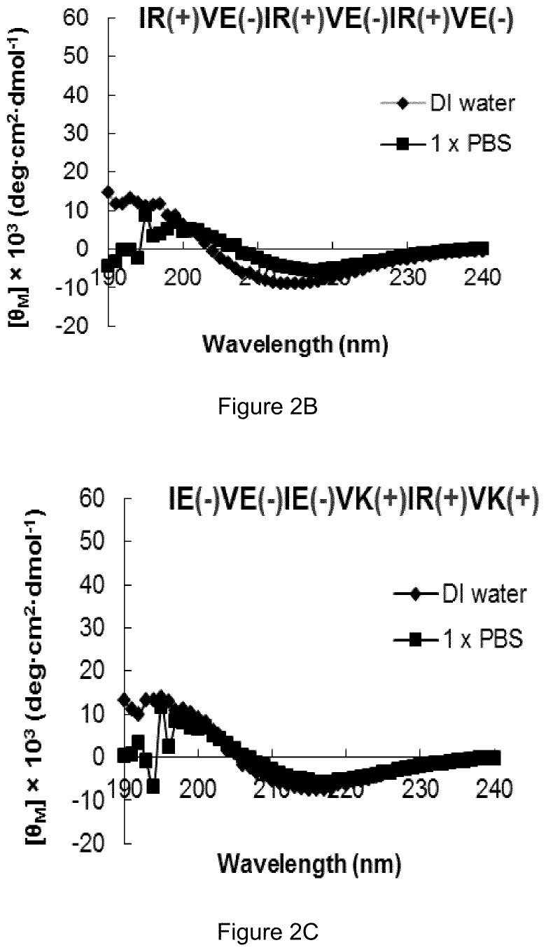

FIG. 2 shows the CD spectra of peptide amphiphiles consisting of 12 amino acids.

FIG. 2A is for IRVKIEVEIRVK (IVK12); FIG. 2B is for IRVEIRVEIRVE (IRV12) and FIG. 2C is for IEVEIEVKIRVK (IEV12). Measurements were carried out at 1.0 mg/mL in water or PBS (pH 7.4) at room temperature (˜23-24° C.).

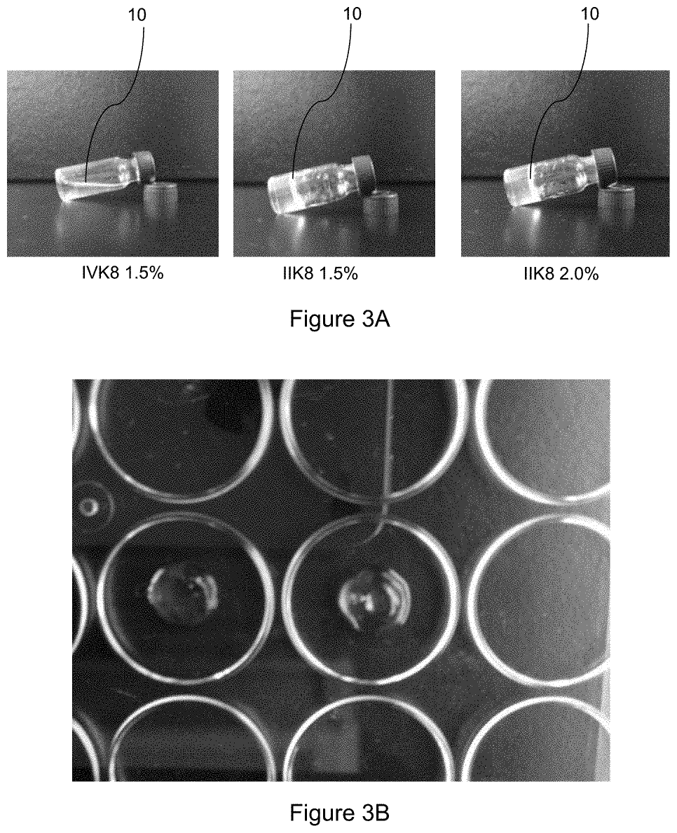

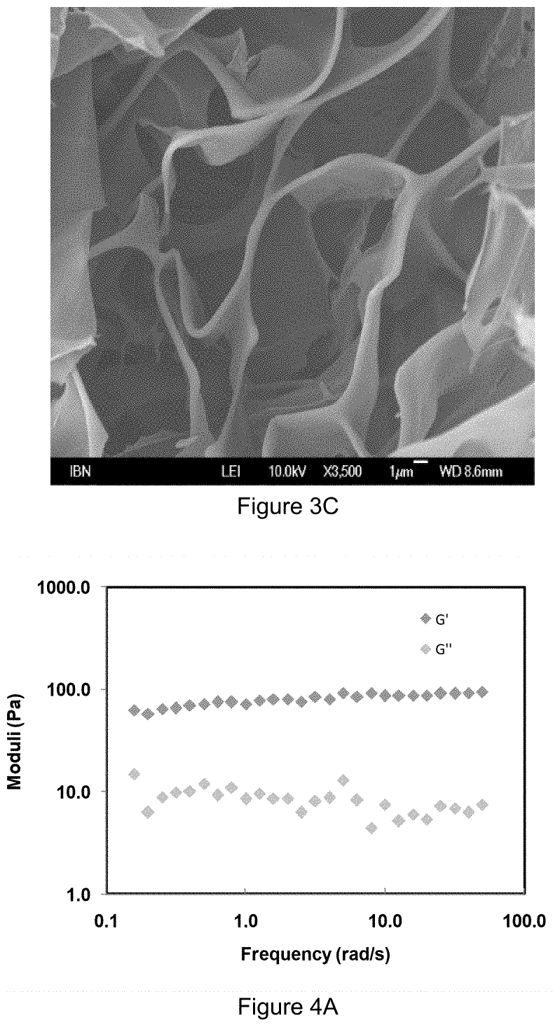

FIG. 3 shows the peptide amphiphiles consisting of 8 amino acids self-assembled into supramolecular hydrogels. FIG. 3A shows how as the hydrophobic amino acid residue Val was substituted with Ile, peptide hydrophobicity increased, leading to faster gelation. Peptides were dissolved in water, incubated at room temperature (˜23-24° C.) for 30 min; FIG. 3B shows the addition of DMEM triggered peptide hydrogel formation; FIG. 3C shows the SEM image of IVK8 hydrogel, which was prepared at a concentration of 1.5% in water and incubated at 37° C. overnight.

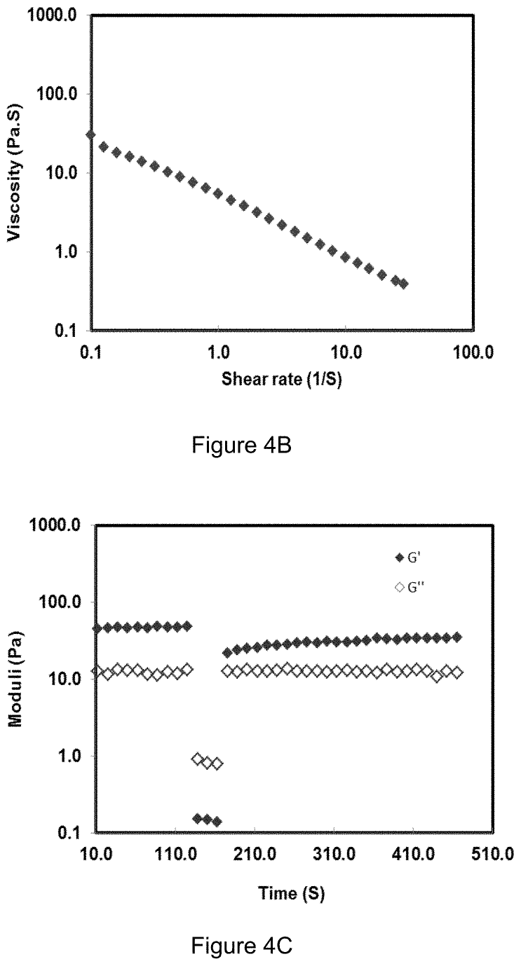

FIG. 4 shows the rheological behaviors of IVK8 in water at a concentration of 1.5 (w/v) %. FIG. 4A shows the effect of frequency sweep of storage moduli (G′) and loss moduli (G″); FIG. 4B shows the flow sweep of viscosity as a function of shear rate; FIG. 4C shows the dynamic step strain amplitude test (strain=0.5% or 100%). The hydrogels were prepared at room temperature (˜23-24° C.).

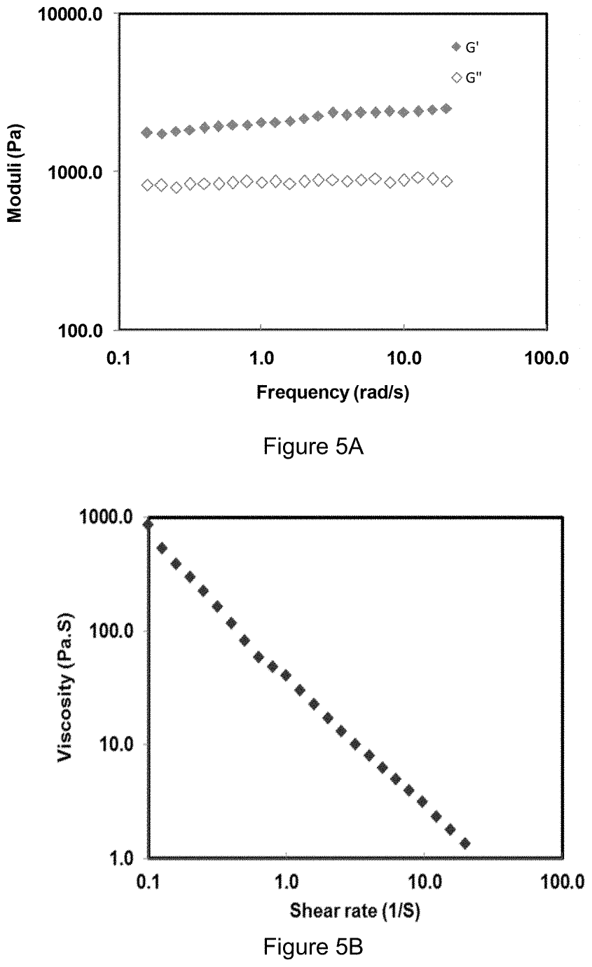

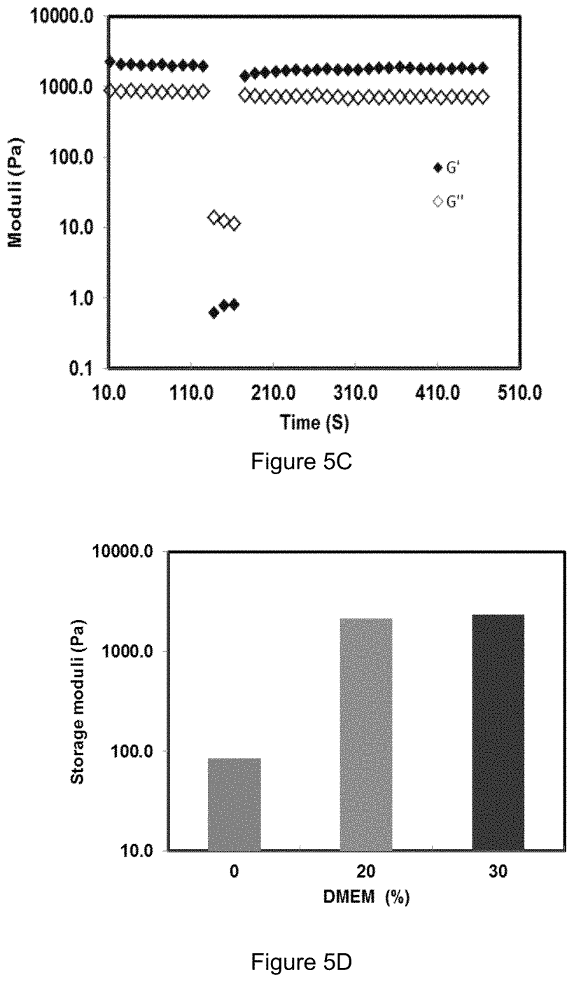

FIG. 5 shows the rheological behaviors of IVK8 in DMEM at a concentration of 1.5 (w/v) %. FIG. 5A shows the frequency sweep of storage moduli (G′) and loss moduli (G″); FIG. 5B shows the flow sweep of viscosity as a function of shear rate; FIG. 5C shows the dynamic step strain amplitude test (strain=0.5% or 100%); FIG. 5D show the effect of DMEM content on the stiffness of peptide hydrogels (Peptide concentrations were 1.5%, 1.2% and 1.0%, for 0, 20 and 30% DMEM respectively). The hydrogels were prepared at 37° C.

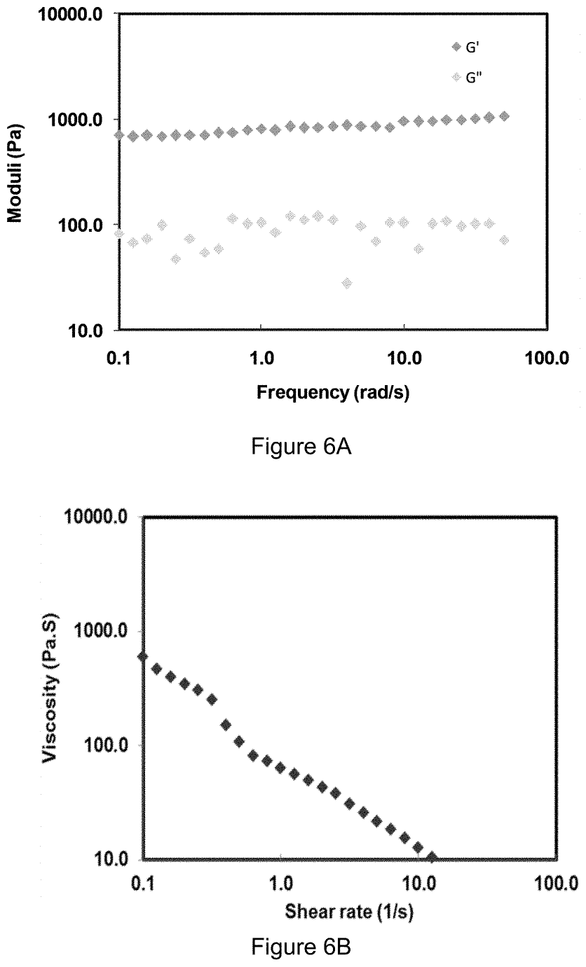

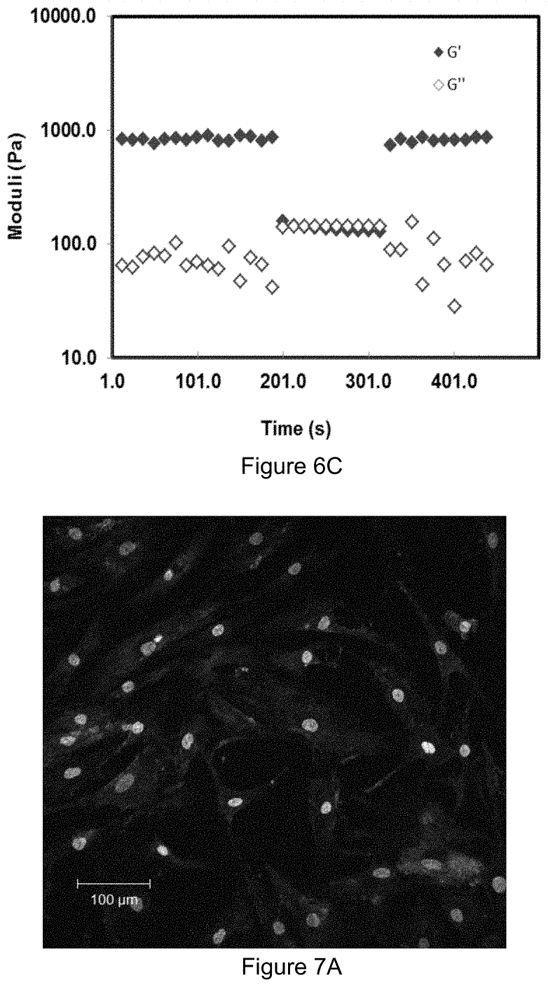

FIG. 6 shows the rheological behaviors of IIK12 (2.0 (w/v) %) in water. FIG. 6A shows the frequency sweep of storage moduli (G′) and loss moduli (G″); FIG. 6B shows the flow sweep of viscosity as a function of shear rate; FIG. 6C shows the dynamic step strain amplitude test (strain=0.5% or 100%). The hydrogels were prepared at 37° C.

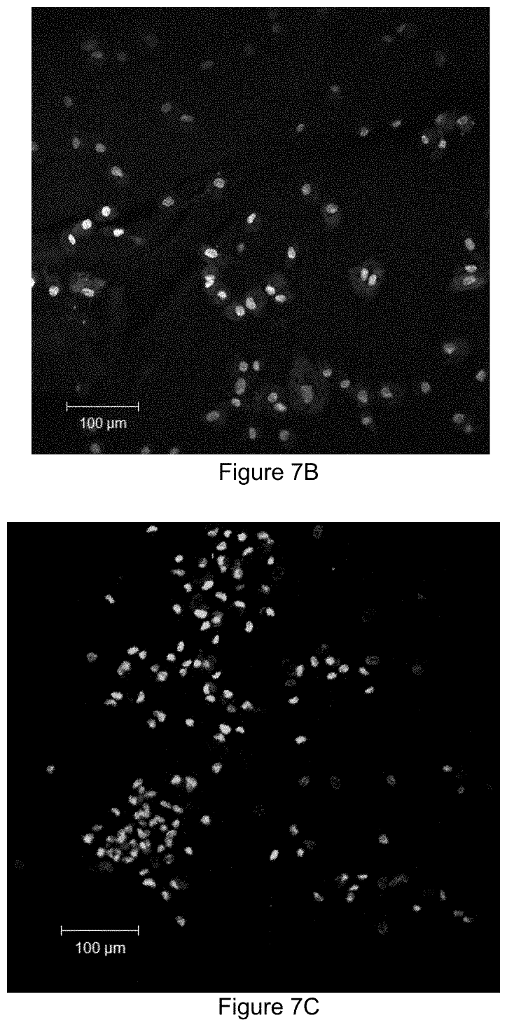

FIGS. 7A-7C shows the confocal images of hMSCs on the surface of IVK8 hydrogels and encapsulation of hMSCs in peptide hydrogels, where cells grown in confocal chamber were used as control. In FIGS. 7A-7C, the live cells are seen as white spots on the black background.

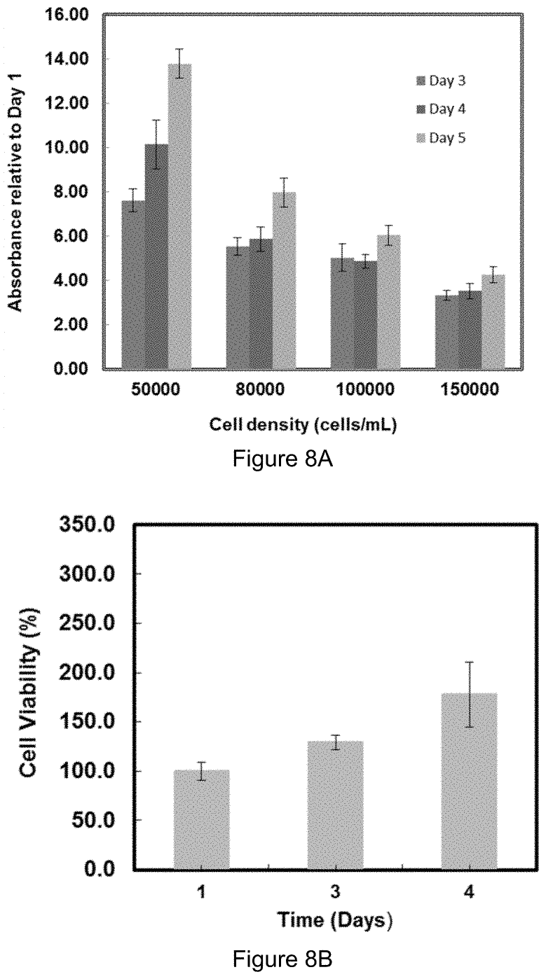

FIG. 8A shows the proliferation of MCF-7 cells on the surface of IVK8 hydrogel and FIG. 8B shows the proliferation of BT-474 cells inside IVK8 hydrogel (3D).

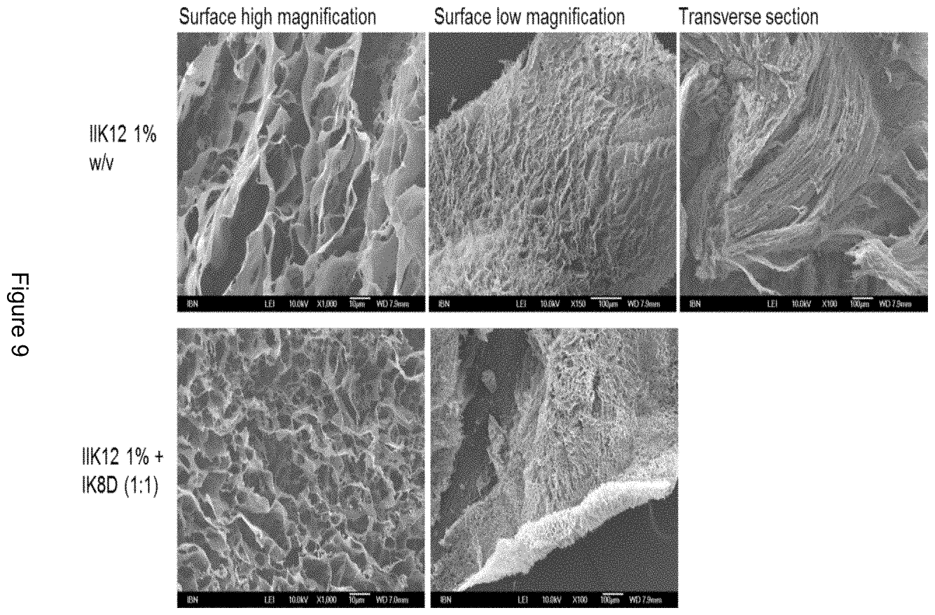

FIG. 9 shows the SEM images of IIK12 and IIK12/IK8D hybrid hydrogels, which were prepared at a concentration of 1.0% of IIK12 in water with and without IK8D (molar ratio of IIK12:IK8D=1:1) and incubated at 37° C. overnight.

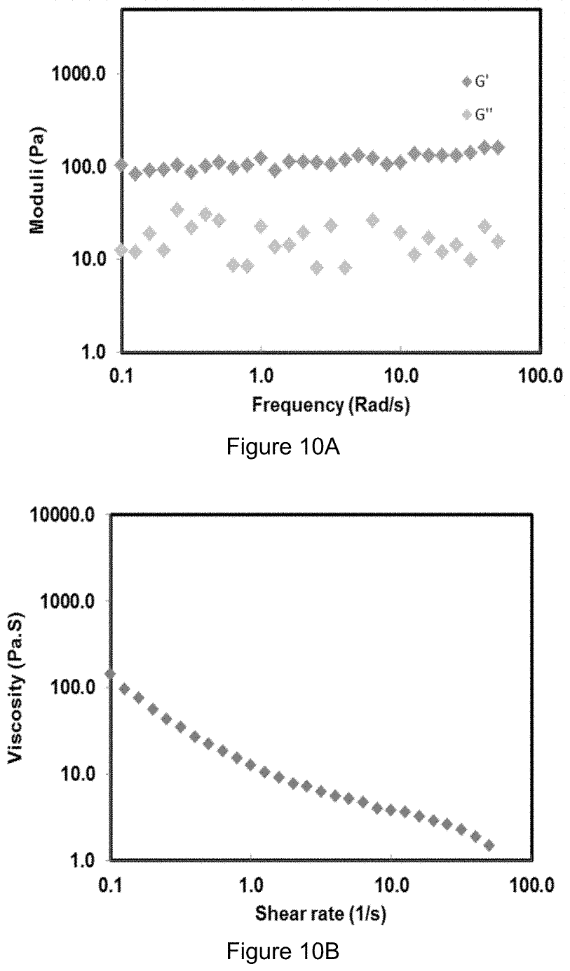

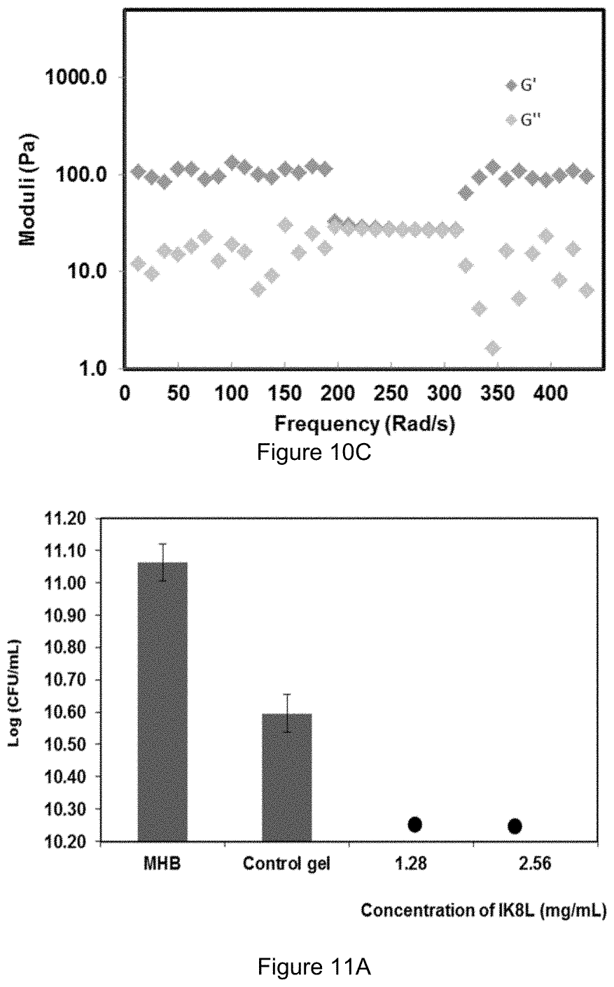

FIG. 10 shows the rheological behaviors of IIK12/IK8L hybrid hydrogel in water at 0.8 (w/v) % of IIK12 and 0.8 (w/v) % of IK8L. FIG. 10A shows the frequency sweep of storage moduli (G′) and loss moduli (G″); FIG. 10B shows the flow sweep of viscosity as a function of shear rate; FIG. 10C shows the dynamic step strain amplitude test (strain=0.5% or 100%). The gels were prepared at 37° C.

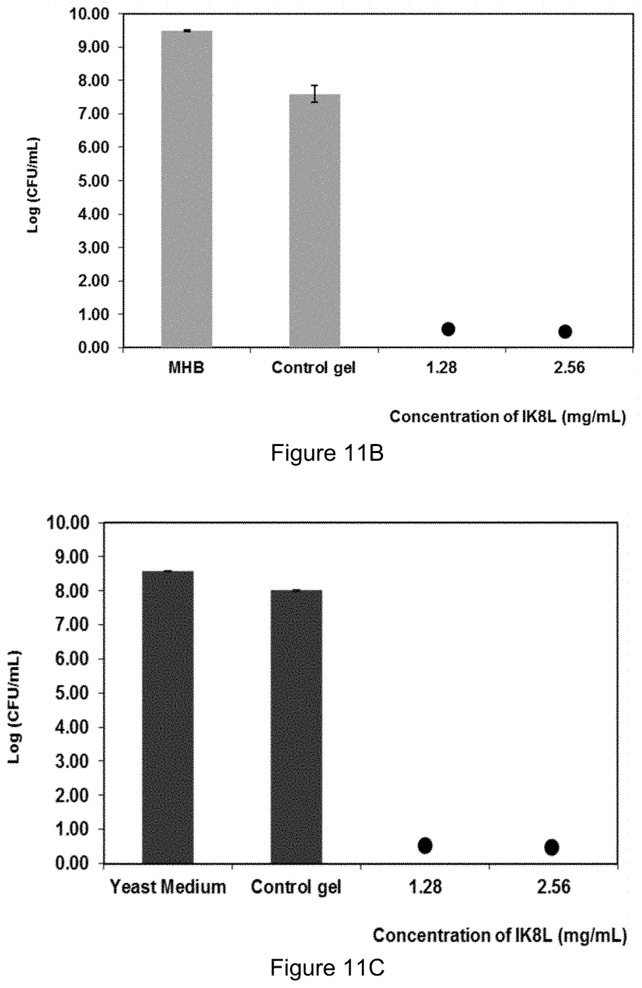

FIG. 11 shows the antimicrobial activity of IIK12/IK8L hybrid hydrogel against various microbes including gram-positive bacteria S. aureus in FIG. 11A, gram-negative bacteria E. coli in FIG. 11B and yeast C. albicans in FIG. 11C. The CFU was calculated at the end of experiments. ⋅ denotes no colonies found.

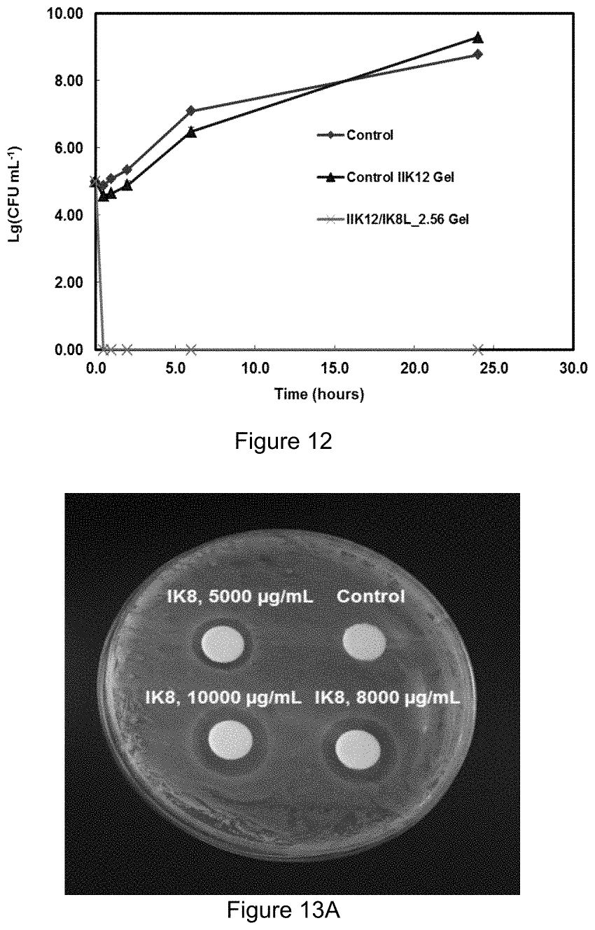

FIG. 12 shows the counts of viable S. aureus cells upon contact with IIK12/IK8L hybrid hydrogels as a function of time. The hydrogels were prepared with IK8L at a concentration of 2.56 mg/mL (i.e. 0.256%). x denotes no colony observed.

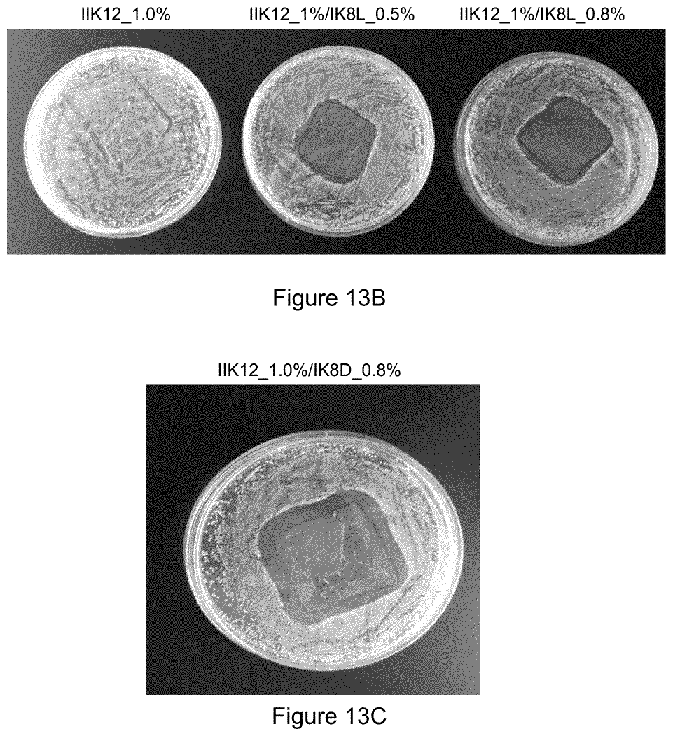

FIG. 13 shows the evaluation of antimicrobial activity of IK8L, IIK12/IK8L and IIK12/IK8D hybrid hydrogels by the disk diffusion assay (DDA). FIG. 13A shows a sterile disk containing IK8L at various concentrations, where water was used as control; FIG. 13B shows a IIK8/IK8L hybrid hydrogel. IIK12_1%/IK8L_0.5% denoted IIK12 at 1% and IK8L at 0.5% in the hydrogel formulation. IIK12 hydrogel was used as control; FIG. 13C shows the IIK8/IK8D hybrid hydrogel, IIK12_1%/IK8D_0.8% denoted IIK12 at 1% and IK8D at 0.8% in the hydrogel formulation. 40 μL of S. aureus suspension were plated on agar plate at 10⁶ CFU/mL. The plates were incubated at 37° C. for 24 h. The lines indicate the area of the applied gel.

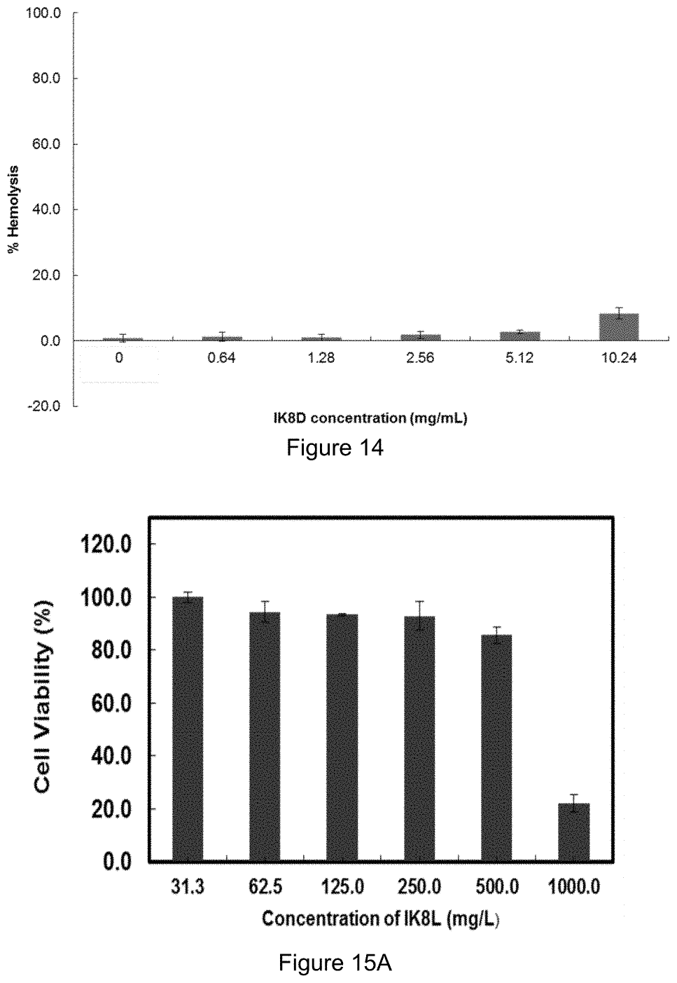

FIG. 14 shows the hemolytic activity of IIK12/IK8D hybrid hydrogel, where the hydrogel was prepared with various concentrations of IK8D. The tests were repeated three times, and the data are expressed as mean±standard deviations.

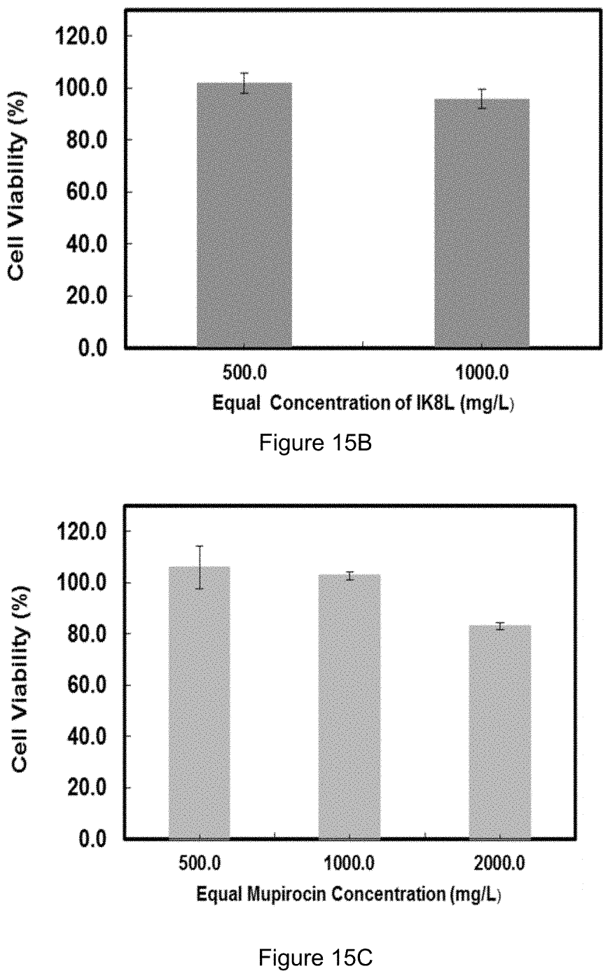

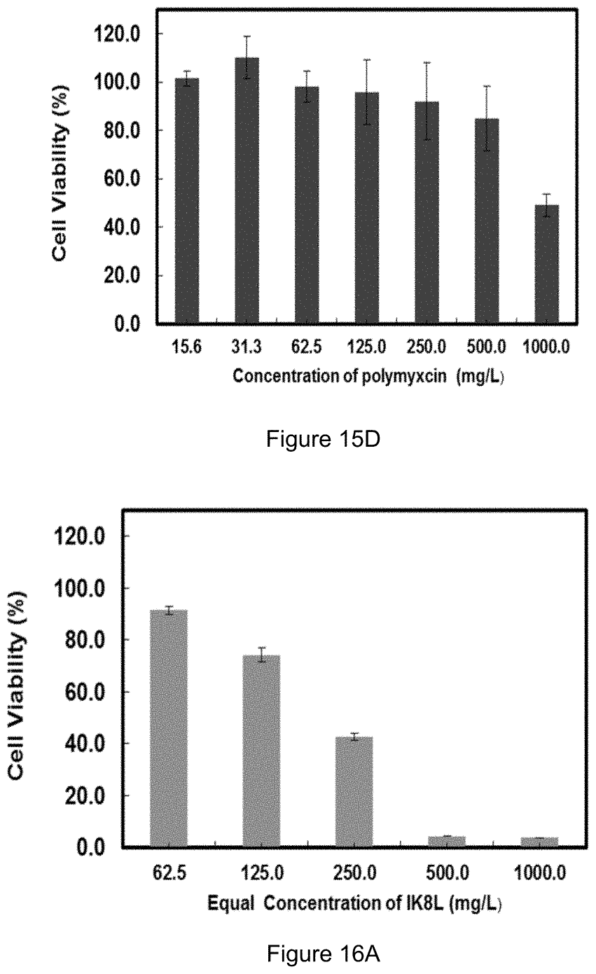

FIG. 15 shows the viability of human dermal fibroblasts after 24 h of treatment with various formulations, which was measured via MTT assay. FIG. 15A shows the viability with the peptide solution of IK8L; FIG. 15B shows the viability with the IIK12/IK8L hybrid hydrogel; FIG. 15C shows the viability with Mupirocin cream; FIG. 15D shows the viability with Polymyxcin B.

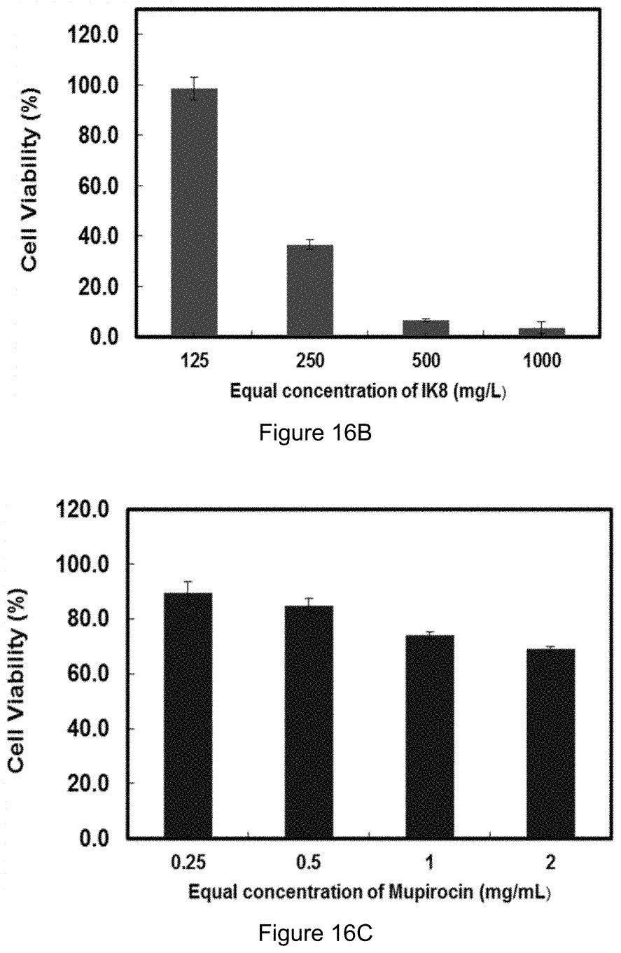

FIG. 16 shows the viability of human primary keratinocytes after 24 h of treatment with various formulations, which was measured via MTT assay. FIG. 16A shows the viability with the peptide solution of IK8L; FIG. 16B shows the viability with the IIK12/IK8L hybrid hydrogel; FIG. 16C shows the viability with Mupirocin cream.

In the following description, numerous specific details are set forth in order to provide a thorough understanding of various illustrative embodiments of the invention. It will be understood, however, to one skilled in the art, that embodiments of the invention may be practiced without some or all of these specific details. Embodiments described in the context of one of the methods or devices are analogously valid for the other methods or devices. Similarly, embodiments described in the context of a method are analogously valid for a device, and vice versa.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The term “agent” and “drug” are used herein, for purposes of the specification and claims, to mean chemical compounds, mixtures of chemical compounds, biological macromolecules, or extracts made from biological materials such as bacteria, plants, fungi, or animal particularly mammalian) cells or tissues that are suspected of having therapeutic properties. The agent or drug may be purified, substantially purified or partially purified.

The term “physiologically acceptable” defines a carrier or diluent that does not abrogate the biological activity and properties of the compound. The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990. The term “physiological conditions” refers to conditions typically found in organisms and cells, and typically refer to conditions with a temperature range of 20-40° C., atmospheric pressure of 1, pH of 6-8, glucose concentration of 1-20 mM, and atmospheric oxygen concentration.

In another aspect, the present disclosure relates to a pharmaceutical composition comprising physiologically acceptable surface active agents, carriers, diluents, excipients, smoothing agents, suspension agents, film forming substances, and coating assistants, or a combination thereof; and a compound disclosed herein. The pharmaceutical composition facilitates administration of the compound to an organism. Acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives, stabilizers, dyes, sweeteners, fragrances, flavouring agents, and the like may be provided in the pharmaceutical composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium methasilicate aluminate, synthetic aluminium silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

Additional therapeutic or diagnostic agents may be incorporated into the pharmaceutical compositions. Alternatively or additionally, pharmaceutical compositions may be combined with other compositions that contain other therapeutic or diagnostic agents.

It will be appreciated that compositions provided herein may be in any form which allows for the composition to be administered to a patient. For example, the composition may be in the form of a solid, liquid or gas (e.g., aerosol). Suitable routes of administration include, without limitation, enteral (e.g. oral, or rectal), topical, parenteral (e.g., sublingually, buccally, sublingual, vaginal, or intranasal). The term parenteral as used herein includes subcutaneous injections, intravenous, intraarterial, intradermal, intramuscular, intrasternal, intracavernous, intrathecal, intraperitoneal, intraocular injections or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units. The compounds can also be administered in sustained or controlled release dosage forms, including depot injections, osmotic pumps, pills, transdermal (including electrotransport) patches, and the like, for prolonged and/or timed, pulsed administration at a predetermined rate.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions suitable for administration include compositions where the active ingredients are contained in an amount effective to achieve its intended purpose. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be between about 10 microgram/kg and 100 mg/kg body weight, preferably between about 100 microgram/kg and 10 mg/kg body weight. Alternatively dosages may be based and calculated upon the surface area of the patient, as understood by those of skill in the art.

The exact formulation, route of administration and dosage for the pharmaceutical compositions of the present invention can be chosen by the individual physician in view of the patient's condition. (See e.g. “Goodman & Gilman's The Pharmacological Basis of Therapeutics” 13^{th} Edition 2017, which is hereby incorporated herein by reference in its entirety,). Typically, the dose range of the composition administered to the patient can be from about 0.5 to 1000 mg/kg of the patient's body weight. The dosage may be a single one or a series of two or more given in the course of one or more days, as is needed by the patient. In instances where human dosages for compounds have been established for at least some condition, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED50 or ID50 values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectables such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In one aspect, administration of a tablet refers to oral administration.

As used herein, the term “immediate release” refers to the attribute indicating that a desired substance is released to its target environment relatively immediately. In one aspect, an “immediate release” tablet releases more than about 40% of the desired substance within hour following administration, as measured under the Tablet Dissolution Test.

As used herein, the term “controlled release” refers to the attribute indicating that a desired substance, such as a drug (e.g., a magnesium salt), is released to its target environment (e.g., a subject) in a controlled fashion, rather than immediately. Thus, a “controlled release” formulation releases no more than about 40% of the desired substance within 1 hour following administration, as measured under the Tablet Dissolution Test. “Controlled release” includes both “delayed release” and “sustained release” formulations. In one aspect, “controlled release” excludes “immediate release” formulations; however, it is contemplated that certain “controlled release” formulations can include an immediate release aspect. For example, a formulation having an immediate release control core and an enteric coating would not be referred to as an “immediate release” formulation; such a formulation can be referred to as a “controlled release” formulation and a “delayed release” formulation, but not as a “sustained release” formulation. Examples of a “controlled release” tablet include a “delayed release” tablet, a “sustained release” tablet, and a “delayed/sustained release” tablet.

As used herein, the term “delayed release” refers to the attribute indicating that a desired substance, such as a drug (e.g., a magnesium salt), is released to its target environment (e.g., a subject) at a time other than promptly after administration. In one aspect, the dosage form controls the drug release rate into the gastrointestinal tract, releasing the bulk of the drug in a portion of the gastrointestinal tract distal to the duodenum. This can decrease the incidence or severity of gastrointestinal side effects. Additionally, this can increase the amount of drug absorbed into the blood. In a further aspect, a “delayed release” formulation releases no more than about 5% of the desired substance within 2 hours following administration. In a yet further aspect, a “delayed release” formulation releases no more than about 5% of the desired substance within 2 hours following administration and releases no more than about 40% of the desired substance within 3 hours following administration. In an even further aspect, a “delayed release” formulation releases no more than about 5% of the desired substance within 2 hours following administration, no more than about 40% of the desired substance within 3 hours following administration, and no more than about 80% of the desired substance within 8 hours following administration. In an even further aspect, a “delayed release” formulation releases no more than about 5% of the desired substance within 2 hours following administration, no more than about 40% of the desired substance within 4 hours following administration, and from about 50 to about 80% of the desired substance within 8 hours following administration. In a further aspect, substantially the entire drug is released within 12 hours. “Delayed release” is a subset of “controlled release”. FDA guidelines also refer to a “delayed release” tablet as a solid dosage form, which releases a drug (or drugs) at a time other than promptly after administration. Enteric-coated articles are delayed release dosage forms. The term includes both “delayed release” tablets and “delayed/sustained release” tablets.

As used herein, the term “delayed/sustained release” refers to the attribute indicating that a desired substance, such as a drug (e.g., a magnesium salt), is released to its target environment (e.g., a subject) at a time other than promptly after administration and released to its target environment in a desired dosage, which is maintained over a desired interval. In one aspect, the dosage form controls the drug release rate into the gastrointestinal tract, releasing the bulk of the drug in a portion of the gastrointestinal tract distal to the duodenum. This can decrease the incidence or severity of gastrointestinal side effects. Additionally, this can increase the amount of drug absorbed into the blood. In a further aspect, the dosage form controls the drug release rate so as to target the distal small intestine. In a yet further aspect, the dosage form controls the drug release rate so as to target the distal small intestine, thereby increasing the amount of magnesium available for interaction with TRPM6 and/or TRPM7 cation channels. In one aspect, the dosage form controls the drug release rate so as to decrease the frequency of dosing. This can maintain desired blood levels of the drug independent of dosing frequency. This can also increase patient compliance with a given treatment regimen. In a further aspect, a “delayed/sustained release” formulation releases no more than about 5% of the desired substance within 2 hours following administration and releases no more than about 40% of the desired substance within 3 hours following administration. In an even further aspect, a “delayed/sustained release” formulation releases no more than about 5% of the desired substance within 2 hours following administration, no more than about 40% of the desired substance within 3 hours following administration, and no more than about 80% of the desired substance within 8 hours following administration. In an even further aspect, a “delayed/sustained release” formulation releases no more than about 5% of the desired substance within 2 hours following administration, no more than about 40% of the desired substance within 3 hours following administration, and from about 50% to about 80% of the desired substance within 8 hours following administration. In a further aspect, substantially of all of the entire drug is released within 12 hours. “Delayed/sustained release” is a subset of “controlled release.” “Delayed/sustained release” is a subset of “delayed release.” “Delayed/sustained release” is a subset of “sustained release.”

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual's physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount,” that is, an amount effective for prevention of a disease or condition.

As used herein, the term “pharmaceutically acceptable carrier” refers to sterile aqueous or non-aqueous solutions, dispersions, suspensions or emulsions, as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminummonostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

The term “immortalized cells” as used herein refers to cells reproduce indefinitely. The cells escape from the normal limitation on growth of a finite number of division cycles. The term does not include malignant cells.

The term “normal cells” as used herein refers to cells that have a limitation on growth, i.e. a finite number of division cycles (the Hayflick limit); therefore, is a nontumorigenic cell. Normal cell include primary cells, which is a cell or cell line taken directly from a living organism which is not immortalized.

The term “cell growth” as used herein refers to an increase in the size of a population of cells.

The term “cell division” as used herein refers to mitosis, i.e., the process of cell reproduction.

The term “proliferation” as used herein means growth and division of cells. “Actively proliferating” means cells that are actively growing and dividing.

The term “inhibiting cellular proliferation” as used herein refers to slowing and/or preventing the growth and division of cells. Cells may further be specified as being arrested in a particular cell cycle stage: G1 (Gap 1), S phase (DNA synthesis), G2 (Gap 2) or M phase (mitosis).

The term “preferentially inhibiting cellular proliferation” as used herein refers to slowing and/or preventing the growth and division of cells as compared to normal cells.

The term “purified” does not require absolute purity; rather, it is intended as a relative definition. Purification of starting material or natural material to at least one order of magnitude, preferably two or three orders, and more preferably four or five orders of magnitude is expressly contemplated. The term “purified” is further used herein to describe a polypeptide or polynucleotide of the invention which has been separated from other compounds including, but not limited to, polypeptides or polynucleotides, carbohydrates, lipids, etc. The term “purified” may be used to specify the separation of monomeric polypeptides of the invention from oligomeric forms such as homo- or hetero-dimers, trimers, etc. The term “purified” may also be used to specify the separation of covalently closed (i.e. circular) polynucleotides from linear polynucleotides. A substantially pure polypeptide or polynucleotide typically comprises about 50%, preferably 60 to 90% weight/weight of a polypeptide or polynucleotide sample, respectively, more usually about 95%, and preferably is over about 99% pure but, may be specified as any integer of percent between 50 and 100. Polypeptide and polynucleotide purity, or homogeneity, is indicated by a number of means well known in the art, such as agarose or polyacrylamide gel electrophoresis of a sample, followed by visualizing a single band upon staining the gel. For certain purposes higher resolution can be provided by using HPLC or other means well known in the art. As an alternative embodiment, purification of the polypeptides and polynucleotides of the present invention may be expressed as “at least” a percent purity relative to heterologous polypeptides and polynucleotides (DNA, RNA or both). As a preferred embodiment, the polypeptides and polynucleotides of the present invention are at least; 10%, 20%, 30%, 40%, 50%, 60%, 70%, 80%, 90%, 95%, 96%, 96%, 98%, 99%, or 100% pure relative to heterologous polypeptides and polynucleotides, respectively. As a further preferred embodiment the polypeptides and polynucleotides have a purity ranging from any number, to the thousandth position, between 90% and 100% (e.g., a polypeptide or polynucleotide at least 99.995% pure) relative to either heterologous polypeptides or polynucleotides, respectively, or as a weight/weight ratio relative to all compounds and molecules other than those existing in the carrier. Each number representing a percent purity, to the thousandth position, may be claimed as individual species of purity.

The terms “polypeptide” and “protein”, used interchangeably herein, refer to a polymer of amino acids without regard to the length of the polymer; thus, peptides, oligopeptides, and proteins are included within the definition of polypeptide. This term also does not specify or exclude chemical or post-expression modifications of the polypeptides of the invention, although chemical or post-expression modifications of these polypeptides may be included or excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. [See, for instance Creighton, (1993), Posttranslational Covalent Modification of Proteins, W.H. Freeman and Company, New York B. C. Johnson, Ed., Academic Press, New York 1-12; Seifter, et al., (1990) Meth Enzymol 182:626-646; Rattan et al., (1992) Ann NY AcadSci 663:48-62]. Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

In this study, we aim to create a new class of short peptides (X₁Y₁X₂Y₂), where X₁ and X₂ are hydrophobic residues (Ile, Val or Leu), Y₁ and Y₂ are hydrophilic residues (Arg, Lys Glu or Asp), n is the repeat unit but it is not necessary for the same amino acid to be selected as is apparent from the examples herein. The peptide amphiphiles self-assemble into hydrogels with tunable mechanical strength and sustainable stability for a range of biomedical applications. To the best of our knowledge, these peptide sequences have not been reported in the literature. Key features of these peptides include regular alternating of hydrophobic and hydrophilic amino acids that encompass both arginine (R) and lysine (K), where the charges are arranged in the pattern (− − + +). R was selected because of its high propensity for gelation while K was employed in combination with R due to its lower cytotoxicity to provide the positively charged moieties at physiological conditions in the peptide. The peptide amphiphiles consisting of 8 amino acid residues exist in a random coil conformation in water and formed peptide hydrogels (˜99% water) at high concentrations. Interestingly, these peptides self-assembled into β sheet conformation and formed stronger hydrogels in the presence of salt or physiological conditions. The hydrophobic residues were systematically tuned to study their effects on the mechanical strength of the peptide hydrogels. The biocompatibility of the peptide hydrogels was evaluated. In addition, another class of short peptide amphiphiles consisting of 12 natural amino acids, IRIKIEIEIRIK (IIK12) and IRVKIEVEIRVK (IVK12), has been designed with alternative hydrophobic and hydrophilic amino acids. The charge arrangement follows the pattern (+ +− − + +), where the central peptide IEIE was flanked by IRIK. IIK12 self-assembled into β sheet conformation and formed hydrogels in aqueous solution at very low peptide concentration (0.6 (w/v) %). It was further complexed with a β sheet-forming antimicrobial peptide (IRIKIRIK (IK8)) through electrostatic interaction, to form a hybrid antimicrobial peptide hydrogel. This hybrid antimicrobial hydrogel is designed for prevention and treatment of DFU infections. The effect of peptide concentration was investigated on the physicochemical properties of the hydrogels. The antimicrobial activity of hybrid hydrogels was evaluated against various pathogenic microbes. Antimicrobial peptide release from the hybrid peptide hydrogels was analyzed using disk diffusion assay.

In addition, it is also possible that peptides with the sequences in the reverse order as that described in the examples would have similar or identical properties, and the terminus of these peptides may be functionalised to avoid forming ions and thus reduce or eliminate electrostatic interaction (especially repulsion) at the terminus, for example the N-terminus may be acetylated and the C-terminus may be amidated. The peptides described herein contain 8 and 12 amino acids, and it is likely that two or more of these peptides may be joined by a linker and is able to form the beta-sheets and hydrogels as described herein. The linker may be any suitable component, length, or moiety and may be joined to the C or N terminus of the peptides by methods known in the art to synthesise peptides of longer length. In addition, peptides with longer amino acid sequences, for example in additional blocks of four amino acids with alternating hydrophobic and hydrophilic amino acids may still be able to form beta-sheets and hydrogels as described herein.

Methods

Materials

The designed peptides were synthesized by GL Biochem (Shanghai, China), and purified to reach 95% purity using analytical reverse phase high-performance liquid chromatography (RP-HPLC). The molecular weights of the synthesized peptides were determined via matrix-assisted laser desorption/ionization time-of-flight mass spectroscopy (MALDI-TOF MS) (Autoflex II, Bruker Daltonics Inc., U.S.A) using saturated α-cyano-4-hydroxycinnamic acid (4-HCCA) (Sigma-Aldrich, Singapore) in acetonitrile/water mixture (1:1 volume ratio) as matrix. 3-[4,5-Dimethylthiazolyl-2]-2,5-diphenyl tetrazolium bromide (MTT) and acetonitrile were purchased from Sigma-Aldrich, and used as received. Human bone marrow mesenchymal stem cells (hMSCs) and mesenchymal stem cell medium (MSCGM) were purchased from Lonza (U.S.A.). Human breast cancer BT-474 cell lines, MCF-7, 4T1 cells were obtained from ATCC, U.S.A., and cultured according to ATCC's recommendation. The cells were cultured in Roswell Park Memorial Institute (RPMI 1640) medium enriched with 10% fetal calf serum, 100 U/mL penicillin and 100 μg/mL streptomycin (HyClone, U.S.A.) at 37° C. with 5% CO₂. Human dermal fibroblasts and human epidermal keratinocytes were obtained from Cordlabs Singapore and cultured according to manufacturer's instruction. Microbe growth medium was prepared using Muller Hinton Broth (MHB) powder (BD Diagnostics). S. aureus(ATCC No. 6538), E. coli (ATCC No. 25922), P. aeruginosa (ATCC No. 9027) and C. albicans (ATCC No 10231) were obtained from ATCC, U.S.A.

Biophysical Studies

CD measurements were performed with a Jasco Model J-810 spectropolarimeter (Jasco, Great Dunmow, Essex, UK) using a quartz cuvette with 1 mm pathlength (Hellma, Germany). Spectra were recorded (average of 3 scans) from 240 to 190 nm at a scan speed of 50 nm/min. The ellipticity was derived from the formula: [θ]_{λ}=(M/(N−1)×θ_{λ})/(d×c), where θ_{λ} represents observed ellipticity (degrees) at wavelength λ, d represents pathlength (cm), c represents the concentration (g/mL), and [θ]_{λ} represents residue ellipticity (deg cm² dmol⁻¹). Thermal scans were measured with Jasco Model J-810 spectropolarimeter equipped with a peltier temperature controller. The solution was allowed to reach equilibrium for 10 min at room temperature prior to testing.

Preparation of Hydrogels

The peptide powder was dissolved in water or other aqueous solutions at the required concentrations. Alternatively, the required salt may be added after the peptide aqueous solution is prepared. The hydrogel was formed by mixing the peptide aqueous solution and salt (if required). For example, the solution was vortexed and then incubated to form hydrogel. The hydrogel formed may be stored as formed, or may be dried, for example by freeze drying. Alternatively, the hydrogel may be formed as required.

The concentration of the solutes (e.g. peptides and salts) herein may be provided in weight by volume percent (and may be abbreviated as (% w/v), (w/v %), or (%) herein, and is equivalent to the mass of solute in grams dissolved in 100 mL of solvent. Alternatively, wt % (or wt. %) may be used to mean the same as the solvent is water and assumed to have a density of 1 g/cm³.

Rheological Studies of Hydrogels

The gelation time was determined by the vial tilting method. When the sample showed no flow, it was regarded as a gel. Rheology experiments were performed at room temperature using a control-strain rheometer (ARES G2, TA instruments). The rheometer is equipped with two sensitive force transducers for torque ranging from 0.05 μN·m to 200 mN·m. The peptide powder was dissolved in water at concentrations ranging from 1.0 to 2.0 (w/v) %. The solution was vortexed and then incubated to form hydrogel. The gel was placed onto parallel-plate geometry (8 mm in diameter). The dynamic storage modulus (G′) and loss modulus (G″) were examined as a function of frequency from 1 to 100 rad/s. The measurements were carried out at a strain amplitude (y) to ensure the linearity of viscoelasticity. Flow sweep (viscosity changes as a function of shear rate) was also performed to study shear thinning properties of hydrogels.

Scanning Electron Microscopy (SEM)

Hydrogels were cut and frozen in liquid nitrogen. The samples were then freeze-dried and the morphology of the hydrogels was observed using a field emission SEM (JEOL JSM-7400F) operated at an accelerating voltage of 10.0 kV and working distance of 8.0 mm.

Culture of hMSCs

hMSCs were cultured in MSCGM medium (Lonza, U.S.A.) and incubated at 37° C., 5% CO₂. The medium was changed every three days. The cells were harvested with PBS containing 0.025 (w/v) % trypsin and 0.01% EDTA, centrifuged and sub-cultured to passage 4 in MSCGM for 2D culture on the surface of hydrogels or 3D hydrogel encapsulation as described below.

Culture of Cells on the Surface of Peptide Hydrogels

IEVEIRVK (IVK8) powder was dissolved in 300 mM sucrose solution at a peptide concentration of 1.5 (W/V) %. It should be mentioned that the sucrose was used to maintain physiologic osmotic pressure. 35 μL of peptide solution was transferred to a 96 well plate. Soft gel was formed followed by the addition of 15 μL of DMEM complete medium. The gel was further solidified for another 10 min of incubation at 37° C. For 2D cell proliferation on the surface of hydrogels, 100 μL of MCF-7 cells in RPMI medium was seeded onto the surface of peptide hydrogel. The culture medium was changed every 2-3 days. The cell proliferation on hydrogels was quantified using MTT assay.

Culture of Cells in 3D Peptide Hydrogels

IVK8 powder was dissolved in 300 mM sucrose solution at a peptide concentration of 1.5 (w/v) % and kept at 4° C. 70 μL of peptide solution was transferred to the open end of 1 mL syringe. It is noted that the open end of the syringe should be cut before the solution transfer. BT-474 cells were suspended in DMEM at 3 million per mL. 30 μL of the cell suspension was added to the peptide solution. The syringe was transferred to an incubator for 20 min incubation at 37° C. After that, the peptide gel was transferred to a 24 well plate for long term culture. The culture medium was changed every 2-3 days. The viability of cells in the hydrogel was quantified by MTT assay. The gels were collected at predetermined time intervals and homogenized with tissue ruptor (Qiagen, U.S.A.). The results were expressed as a percentage of the absorbance at Day 1.

Confocal Laser Scanning Microscopic Studies

The morphology and viability of hMSCs on the surface of hydrogels and inside 3D peptide hydrogel environment was observed by an inverted confocal laser scanning microscope (CLSM) (CLSM, Carl Zeiss, Germany). Live/Dead cell staining assay (Invitrogen) was used. Briefly, cells cultured on the surfaces of hydrogels in chambered cover glass (NUNC, Denmark) were washed with PBS and incubated in 0.5 mL of PBS containing dyes for 10 min, then visualized by CLSM at excitation wavelengths of 488 nm and 532 nm. All the observations were conducted under the same conditions. For the cells grown in 3D hydrogels, the constructs were cut, followed by the addition of staining agent. The gels were transferred to cover glass and then visualized by CLSM using the same method as described for the cells on the hydrogel surface.

Antimicrobial Activity

1. MIC Measurement for Peptides

Bacterial and fungal samples were grown in MHB at 37° C. and room temperature, respectively, under shaking (100 rpm). They were subsequently incubated overnight so as to enter the log growth phase. The respective MIC of each peptide was determined using a broth microdilution method. Each microbial suspension (100 μL) was seeded into each well of a 96-well plate (3×10⁵ CFU ml⁻¹), to which 100 μL of broth containing a peptide at different concentrations was added. The plate was then subjected to incubation under shaking (100 rpm) for 18 h at 37° C. MIC was taken to be the lowest peptide concentration at which microbial growth was completely inhibited by observation with a microplate reader (TECAN, Switzerland). Negative controls (broth containing only microbes without peptide treatment) were used. Six replicates were repeated for each experiment. It is noted that C. albicans cells were grown in yeast medium broth (YMB, BD) at room temperature.

2. Antimicrobial Activity of Peptide Hydrogels

Hydrogels for the antimicrobial assays were prepared in 96-well tissue culture plates (NUNC). Briefly, IIK12 (1%) with various contents of antimicrobial peptides was dissolved in water and vortexed. 50 μL of solution was transferred to the wells. Gelation occurred overnight at 37° C. The growth of microorganisms on hydrogels was measured using a broth dilution method. Briefly, 30 μL of microorganism suspension (3.5×10⁵ CFU/mL) was introduced onto the hydrogels, and a 96-well plate and peptide hydrogel without antimicrobial peptides were used as controls. The optical density readings of bacterial solutions were monitored by measuring OD_{600 nm}. The assay was performed in four replicates for each sample and the experiments were repeated at least three times. Antimicrobial activity was further tested through a spread plate method. Briefly, hydrogels were challenged with microorganism solution, at the end of treatment, microbial suspension on the hydrogel surface was withdrawn and diluted sequentially, and then plated on 1.5% LB agar plates. The plates were incubated for 24 h at 37° C. (48 h at room temperature for C. albicans). Microbial colonies were formed and counted. The experiments were performed in triplicate and were repeated three times.

3. Killing Kinetics Test

The microbes were treated with peptide hydrogels as described above. At various time periods of treatment, the microbial samples were diluted for plating (LB Agar, 1st Base). Incubation conditions: 18 h, 37° C. for bacteria. The colony-forming units were counted after the incubation period. The experiment was repeated in independent settings three times.

4. Disk Diffusion Assay

Antimicrobial activity was further tested through a disk diffusion assay. Briefly, microbial suspension (S. aureus, ATCC No. 6538) with 3.5×10⁶ CFU/mL (40 μL) was spread on 1.5% LB agar plates. Disks (Sigma) were prepared to contain 50 μL of peptides with various concentrations by dripping 50 μL of peptides solutions onto a sterile disk. Disks were air-dried for several minutes before being placed onto different zones in the agar plates. Hydrogels (100 μL) were placed onto LB agar plates. The plates were incubated for 24 h at 37° C.

5. Hemolysis Assay

Fresh rat red blood cells were washed 3 times with PBS. 100 μL of red blood cell suspension in PBS (4% in volume) was placed on the surface of hydrogel in each well of a 96-well plate and 100 μL of PBS was then added to the well. The plates were incubated for one hour at 37° C. The 96-well plates were centrifuged at 2000 rpm for 5 min. Aliquots (100 μL) of the supernatant were transferred to a new 96-well plate. Hemoglobin release was measured at 576 nm using a microplate reader (TECAN). The red blood cells in PBS were used as a negative control. Absorbance of wells with red blood cells lysed with 0.2% triton X-100 was taken as 100% hemolysis. Percentage of hemolysis was calculated using the following formula: Hemolysis (%)=[(OD_{576 nm} in the sample−OD_{576 nm} in PBS)/(OD_{576 nm} in 0.2% triton X-100−OD_{576 nm} in PBS)×100. The data were expressed as mean and standard deviation of four replicates and the tests were repeated 3 times.

6. In Vitro Cytotoxicity Study of Antimicrobial Peptide Hydrogels

1000 μL of HDFs or keratinocytes (10⁵ cells per mL) were seeded onto the 12-well Transwell plates (Corning). IIK12 (1%) with various contents of antimicrobial peptides were dissolved in water and incubated at 37° C. overnight and 100 μL of gels were then transferred to the inserts of the Transwell plate. 1 mL of fresh medium was added into the inserts. The plates were incubated for 24 h before proceeding to MTT assay. It is noted that cells without any treatment and Mupirocin (GSK) were used as controls. The data were expressed as mean and standard deviation of three replicates and the tests were repeated 3 times.

Results and Discussion

Peptide Design and Characterization

In this study, short amphiphilic peptides consisting of 4-12 natural amino acid residues were designed. It is preferred for the peptides to have an even number of amino acid residues to maintain the chemical complementarity of the peptide. These peptide sequences are characterized by periodic repeats of charged hydrophilic and hydrophobic amino acids, where the hydrophobic residues are isoleucine (I), valine (V) and leucine (L), and the hydrophilic residues are arginine (R), lysine (K), glutamic acid (E) and aspartic acid (D). Arginine and lysine are positively charged at physiological conditions or a pH of 7 to 7.4 at 25° C., while glutamic acid and aspartic acid are negatively charged. Isoleucine (I), valine (V) and leucine (L) were selected as hydrophobic residues as they have strong β-sheet folding propensity. Notably, arginine (R) was selected because of its faster and stronger gelation property while lysine (K) was used in combination with R due to its lower cytotoxicity. To minimize charge repulsion, the peptides were amidated at the C terminus. As the N-terminus has a hydrophobic residue, it is not necessary to acetylate the N-terminus, but may be done by known methods. If the N-terminus has a hydrophilic residue, it may be functionalized by known methods for example acetylation to minimize charge repulsion. RP-HPLC showed that the purity of the peptides was greater than 95%. MS was used to verify the molecular weight of the peptides. As shown in Table 1, the measured molecular weights via MS are in agreement with the theoretically calculated molecular weights of the peptides, indicating that the peptides were successfully synthesized.

In addition, it is likely that peptides with the sequences reverse to SEQ ID No. 9-16 are able to function similarly, substantially similarly or identical to the corresponding hydrogel. These peptides are identified in Table 1 as SEQ ID No. 19-26 respectively, and may be prepared by methods known in the art. These peptides with the reverse sequence should preferably be acetylated at the N-terminus and amidated at the C-terminus. This minimises charge repulsion on both ends of the peptide.

Peptides containing both positively and negatively charged amino acids may be able to form hydrogels at lower concentrations than peptides with only positively charged amino acids. For example, peptides with only positively charged amino acids generally form gels at a concentration of 8% w/v or higher, whereas peptides with both positively and negatively charged amino acids form hydrogels from 0.6% w/v and higher. In addition, peptides with only negatively charged amino acids do not undergo self-assembly to form hydrogels.

CD Studies of the Peptides

As shown in FIG. 1A, the CD spectrum of IVK8 in aqueous solution displayed a minimum at 198 nm, indicating that it adopted a random coil structure. This was attributed to the intermolecular electrostatic repulsion between the protonated R and K residues. However, in the presence of salt such as PBS, IVK8 folded and self-assembled into β-sheet conformation which was characterized by a maximum at ˜197 nm and strong negative ellipticity at ˜219 nm. Salt triggers self-assembling of the peptide because it screens and neutralizes the charges, where hydrophilic residues E, R and K lied on the same side with complementary ionic interactions derived from positively and negatively charged ionic groups. Similar observations were also reported by Zhang et al., where EAK16 and EAK12 self-assembled into β-sheet secondary structures upon the addition of salt[17, 35]. However, EAK8 (AEAEAKAK) did not form β-sheet secondary structures under the same conditions[35]. Unlike EAK8, our peptide IEVEIRVK (IVK8) self-assembled into β-sheet secondary conformation upon the addition of salt. A possible explanation is that the use of hydrophobic isoleucine and valine on the hydrophobic peptide face increased peptide hydrophobicity, which in turn stabilized the β-sheet conformation of IVK8 through enhanced hydrophobic interaction between isoleucine and valine. Based on the above studies, IVK8 was identified as the peptide with the ideal length and sequence, and therefore its hydrophobicity was further varied to study its effect on biophysical properties. The substitution of Val with Leu or Ile in IVK8 yielded peptide sequence ILK8 and IIK8, respectively. Similar to IVK8, ILK8 and IIK8 adopted a random conformation in aqueous solution but folded into β-sheet structures with the addition of salt (FIGS. 1A and B). Interestingly, the substitution of Val with Leu or Ile resulted in greater extent of β-sheet folding, which is indicated by the negative values of the molecular ellipticity (θ_{M}) at the minimum points. It can be seen that the θ_{M} were −11.2, −21.4 and −28.9 deg.cm²dmol⁻¹ for IVK8, ILK8 and IIK8, respectively, indicating that IIK8 had the greatest β-sheet conformation as compared to ILK8 and IVK8. This is because Ile and Leu are more hydrophobic as compared to Val, which increased the peptide hydrophobicity on the non-polar side and leading to greater stabilization of the β-sheet and increased peptide β-sheet conformation[28, 36]. It is a general belief that peptides of alternating hydrophilic and hydrophobic amino acid residues with more repeat units tend to have high propensity in forming β-sheet conformation. Therefore, a new class of peptide amphiphiles composed of (X₁Y₁X₂Y₂)₃ with 12 amino acids were designed, where X₁ and X₂ are hydrophobic residues (Ile or Val), Y₁ and Y₂ are hydrophilic residues (Arg, Lys or Glu) (Table 1). Interestingly, these peptide amphiphiles fold and assemble into a β-sheet structure in water, as illustrated by the strong negative ellipticity at 218 nm (FIG. 2). The CD spectra of peptide amphiphiles consisting of 12 amino acids were further investigated in the presence of salt. The minimum negative ellipticity at 218 nm for the peptide solution with 150 mM PBS indicated that they also adopted secondary structures rich in β-sheet. It is noted that the β-sheet content for peptide amphiphiles IVK12 in the presence of salt is similar to that in the absence of salt (FIG. 2A). This is because IVK12 has a net positive charge (+2) at PBS. Electrostatic repulsion led to the low degree of β-sheet conformation for peptide amphiphile IVK12. A similar phenomenon was also observed for IRV12 and IEV12 (FIG. 2). Although IRV12 and IEV12 have different arrangement of the charged species, they have similar net charges and display similar gelation behavior, thus it is likely the net charge may play a more important role than the specific arrangement of the hydrophilic residues. Advantageously, a peptide with a net positive charge may form the beat sheets and hydrogel more readily even in the absence of salts, which may be necessary for certain applications of the hydrogel and also leads to lower costs.

Rheological Properties of Peptide Hydrogels

It is generally recognized that peptide amphiphiles have a strong tendency to forma-helix or β-sheet structures. Under the appropriate conditions, the resulting α-helices or β-sheets can further self-assemble into supramolecular structures such as hydrogels entrapping a large amount of water (FIGS. 3A and 3B)[16]. A typical SEM image of hydrogels was illustrated in FIG. 3C. It is clearly seen that the hydrogel is highly porous. Peptide gelation can be tuned by modulating intrinsic factors such as amino acid sequence, number of repeating units, peptide concentration and external environments such as temperature, pH and salt concentration[23].

Effects of Peptide Length and Peptide Structure

Peptide amphiphiles consisting of 4-6 amino acid residues could not form hydrogels at concentrations up to 2% (Table 2). These results are in agreement with previous findings that peptide length dictates the strength of intermolecular and intramolecular interactions. Peptide amphiphiles comprising 8 amino acid residues have been designed as listed in Table 2. Firstly, the positions of hydrophilic ion residues have been systematically tuned to evaluate their effects on peptide gelation. There is no gel formation (up to 2%) for IRK8, where hydrophilic amino acid residue are arranged in the fashion of (+ − − +). It may be possible that IRK8 may form a gel at a higher temperature (for example 37° C.) and/or concentration. However, gelation was observed for IRE8, where hydrophilic amino acid residue are arranged in the pattern of (+ − + −). It should be mentioned that the gelation kinetics is slow, for example, it took 3 days for IRE8 to form the gels with concentration of 2% at room temperature. The gelation time was reduced significantly to 1 day when the temperature increased to 37° C. Similarly, it is expected that the 12 amino acids peptides may form a hydrogel at room temperature with prolonged gelation time and/or higher concentrations.

Notably, fast gelation was achieved for IVK8, where hydrophilic amino acid residues are arranged in the pattern of (− − + +). For instance, at 2%, gelation of IVK8 occurred within 60 min at room temperature (23-24° C.), while it formed gel within 30 min at 37° C. Using IVK8 as a basic peptide sequence, its hydrophobic amino acid residues Val were replaced with either Ile or Leu, while the hydrophilic amino acid residues and arrangements remained unchanged. The substitution of Val with Ile or Leu led to faster gelation under the same conditions (Table 2). For instance, gelation occurred for IIK8 within 15 min at 1.5% when incubated at 37° C. It was also observed that the increase in peptide length to 12 amino acid resulted in lower gelation concentrations. Notably, IIK12 and IVK12 are the best performing peptides because of easy gelation, which self-assembled into hydrogels at concentrations of as low as 0.6 (w/v) %. These findings suggested that peptide chain length and peptide structure play an important role in gelation. In FIG. 3A, the hydrogel formed from 1.5% w/v IVK8 and 1.5 and 2.0% w/v IIK8 are shown with the white band being the surface 10 of the gel in the vial.

The speed of gel formation may be increased by adjusting at least one of the following: peptide concentration, presence of a salt, and increasing the temperature. Peptides with 12 amino acid residues generally form the gel more readily compared to the peptides with 8 amino acids. Further, peptides with a net positive charge (but containing negatively charged hydrophilic amino acids) tend to form the hydrogels at lower concentrations due to enhanced beta sheets hydrogen bonds.

Effects of Peptide Concentration and Peptide Hydrophobicity on Mechanical Property

Effects of peptide concentration on the rheological properties were investigated using dynamic mechanical analysis. Peptide amphiphiles self-assembled into supramolecular hydrogels exhibiting rigid viscoelastic properties. A typical frequency sweep measurement was illustrated in FIG. 4A and FIG. 5A, where storage modulus (G′) was much greater than the loss modulus (G″) values. Moreover, the G′ values were independent of the frequency ranging from 0.1 to 50 rad/s. The G′ values at 1 Hz (6.28 rad/s) were listed in Table 3 and used for gel stiffness comparison. It was found that increasing peptide IVK8 concentrations from 1.0% to 1.75% increased the stiffness significantly. For instance, the G′ value at 1.75% is 10-fold higher than that at 1.0% (Table 3). The results suggested that peptide gel stiffness was strongly affected by peptide concentration (i.e. amount of peptide in the aqueous solution). This is because more peptide molecules participated in physical cross-linking at higher concentrations. To investigate the effects of hydrophobicity of amino acid residues on gel strength, all the Val within IVK8 were substituted by Ile or Leu. This modifications yielded peptide IIK8 and ILK8. The G′ values of IVK8, IIK8 and ILK8 were 84, 125 and 332 Pa, respectively (Table 3), indicating that increasing hydrophobicity of non-polar amino acid residue in the peptide structure led to stiffer gel. It has been reported that Ile and Leu had similar hydrophobicity. However, ILK8 formed gel with greater strength as compared to that of IIK8. This is because Leu may pack more efficiently than Ile during the self-assembling and gelation process. The maximum peptide concentration is likely to be about 3-5 w/v %, at most 5 w/v %, at most 4 w/v %, or at most 3 w/v %. The presence of excess peptides may make the hydrogel too stiff for certain applications and the optimal peptide concentration would depend in part on the peptide structure and use of the peptide. This may be further adjusted by the presence of a salt as shown below.

Effects of Salt

In aqueous solution, peptide amphiphiles consisting of 8 amino acids existed in a random coil conformation. However, it self-assembled into β-sheet structure and formed stiff hydrogels upon the addition of salt. For instance, in the presence of PBS, IVK8 had a G′ of ˜900 Pa at 1.5%, which is nearly 2-fold of that in the absence of salt. Similar phenomena were observed in DMEM. When DMEM cell culture media was added to aqueous peptide solution, the peptide formed a rigid hydrogel. For instance, in DMEM, IVK8 had a G′ of ˜2400 Pa at 1.5%, which is nearly 5-fold of that in the absence of DMEM. Notably, in PBS, an extremely rigid hydrogel of 10 kPa was obtained for IVK8 at 2.5%. In particular, fast gelation was achieved at 37° C. This is critical for tissue engineering applications as gelation kinetics must be fast enough to ensure homogeneous cell distribution within the matrix. In addition, stiffer gels were formed at 37° C. than the room temperature (Table 3).

Moreover, the gel was obtained with high stability in cell culture environment under optimal conditions (by tuning peptide concentration and DMEM volume) (Table 4). Table 4 shows the initial concentration of IVK8 used (1.0, 1.2 and 1.5% w/v) which was adjusted by the addition of DMEM to achieve the ratios shown in Table 4. It may be observed that the hydrogels prepared were stable for at least 7 days for most of the hydrogels and at least 15 days for the higher concentration peptide hydrogel.

The dramatic increase in peptide hydrogel's strength in the presence of salt was attributed to the fact that the salt screens the charges and promotes intramolecular and intermolecular interactions (hydrogen bonding and Van der Waals interactions), resulting in highly crosslinked network. It will be ideal if the hydrogel construct containing drugs and/or cells can be injected to the body and remain intact at the point of administration. For peptides with 8 amino acids, the presence of a salt in the solution increases the stiffness of the hydrogel and decreases the concentration of the peptide required to form the gel. Without the presence of a salt, it has been found that IVK8, IIK8 and ILK8 form the gel at a minimum concentration of 1.0% w/v. In the presence of a salt, the concentration for these peptides can decrease to 0.6% w/v leading to lower costs to prepare the gel. Further, the salt greatly decreases the gelation time be it at room temperature or at higher temperatures (e.g. 37° C.).

The flow sweep was performed to investigate whether the peptide hydrogel is injectable. FIG. 4B shows that viscosity of IVK8 is reduced significantly with increasing shear rate, indicating shear-thinning property of the hydrogels. A similar phenomenon was also observed for IVK8 in the presence of DMEM (FIG. 5B). Gelation of the IVK8 peptide solution was triggered with the addition of DMEM, where DMEM was in the network of peptide hydrogel.

The reduction of viscosity was attributed to the disruption of physical cross-links between the peptide molecules upon the application of shear stress. In order for a peptide gel construct containing a drug and/or cell/s to be delivered by syringe, it is essential that the disrupted gel with low viscosity could rapidly recover back into a stiff gel when the shear stress terminates[26]. The ability of the peptide hydrogels to recover was investigated using a dynamic step strain amplitude test, where strain of 0.5% and 100% were periodically applied on the peptide hydrogels. As illustrated in FIG. 4C, initial G′ value of IVK8 was ˜50 Pa when the strain of 0.5% was applied. However, when subjected to a high strain (100%), the G′ value rapidly decreased by ˜250-fold to 0.2 Pa, further demonstrating shear-thinning property. After subjected 30 s of continuous high strain, the strain was reduced to 0.5%. Interestingly, it was found that the G′ value was recovered immediately, indicating reversible rheological behavior. This reversibility of peptide hydrogel in the presence of DMEM was further evaluated under the same conditions. FIG. 5C shows initial G′ value was ˜2000 Pa when the stain of 0.5% was applied. The G′ value is reduced remarkably by ˜2000 times to ˜1 Pa when subjected a high stain of 100%. Notably, the G′ value is recovered immediately when the strain was decreased to 0.5%. A similar phenomenon was also observed for IIK12 hydrogel, where the storage moduli of IIK12 hydrogel increased with increasing peptide concentrations (Table 5). Moreover, IIK12 hydrogel demonstrated reversible rheological behavior in response to a dynamic step strain amplitude measurement (FIG. 6). This rheological behavior of the peptide hydrogels offers a great advantage for use as an injectable matrix for delivery of therapeutics such as drugs and cells. The fast recovery kinetics can be explained by the fact that the physical cross-links of the gel network immediately recovered after the high strain terminated.

In Vitro Biocompatibility of Peptide Hydrogels

The biocompatibility of the peptide hydrogels and their effect on cell proliferation were investigated using different cell types. Firstly, hMSCs were seeded on the surface of peptide hydrogels. As shown in FIG. 7, the majority of the cells remained viable (the live cells are seen as white spots on the black background) after 24 h attachment on the surface of peptide hydrogel, indicating that the peptide hydrogels are biocompatible. Next, we analyzed the proliferation rate of MCF-7 cells on IVK8 hydrogels. As shown in FIG. 8A, the proliferation rate depended on cell seeding density. The MCF-7 cultured on peptide hydrogels with lower cell density grew much faster than the ones with higher cell density. This is because cell growth was restricted by the limited space with 2D culture. Fortunately, this can be overcome by cell culture in 3D environment. Due to the ability of entrapping high water content, hydrogels are attractive matrix for cell growth in a 3D environment. Moreover, hydrogels have high permeability of oxygen, nutrients through their high water content matrix, which is desirable for cell growth and tissue engineering. Peptide was dissolved in aqueous solution with 300 mM sucrose and formed vicious solution at 4° C. Gelation was triggered with the addition of cell culture medium containing hMSCs. Cellular viability of encapsulated hMSCs was assessed to evaluate the cytotoxicity of the peptide hydrogels. Viability was evaluated by live/dead stain using a confocal microscope 1 day after encapsulation. It was found that the majority of encapsulated hMSCs remained viable in IVK8 hydrogels. BT-474 cells were also encapsulated in IVK8 hydrogels and cell growth was quantified using MTT assay. FIG. 8B showed that cell number in the hydrogels at day 4 was almost one fold higher than that at day 1, indicating that peptide hydrogels is a suitable matrix for the 3D culture of cells. Taken together, injectable peptide hydrogels with tunable mechanical properties offer a promising biocompatible system for cell growth and tissue engineering.

Peptide Hydrogels Containing β-Sheet Forming Synthetic Antimicrobial Oligopeptides for Prevention and Treatment of Bacterial Infection

We have previously developed a series of β-sheet forming synthetic cationic oligopeptides that demonstrate broad-spectrum antimicrobial activity and high selectivity towards various microbes including Gram-positive S. aureus, Gram-negative E. Coli and P. aeruginosa and yeast C. albicans[37, 38]. The peptide formed from D-amino acids exhibited more potent antimicrobial activity. The leading antimicrobial peptides, IK8L (IRIKIRIK SEQ ID No.17) and IK8D (D-enantiomer, SEQ ID No. 18) effectively killed various clinically isolated multi-drug resistant (MDR) microorganisms including MRSA, VRE, multidrug-resistant A. baumannii, P. aeruginosa and yeast C. neoformans [37]. For instance, the MICs of IK8L against MRSA, A. baumami, VRE, P. aeruginosa and C. neoformans were 31.3, 12.5, 15.6, 7.8 and 7.8 mg/L, respectively. The MICs of IK8D against MRSA, A. baumannii, VRE, P. aeruginosa and C. neoformans were 3.9, 31.3, 3.9, 15.6 and 7.8 mg/L, respectively. In particular, repeat treatment of E. coli and S. aureus with the peptides mitigating drug resistance development. IK8L also demonstrated strong anti-fungi activity, and is effective in removing fungal biofilms formed both in vitro and in a fungal biofilm-induced keratitis mouse model without causing significant toxicity to the eyes[39]. However, IK8D/IK8L forms a hydrogel at higher peptide concentrations (for example 8 w/v %), which will induce cytotoxicity at the gelation concentration making them unsuitable for therapeutic use alone. However, the addition of these antimicrobial peptides to IIK12 hydrogel through electrostatic interaction forms a hybrid antimicrobial peptide hydrogel. We systematically evaluated the antimicrobial activity of the hybrid hydrogels against pathogenic microbes.

Rheological Behaviour of Antimicrobial Hybrid Hydrogels

Table 6 shows gelation of 0.5% IIK12 and IK8L or IK8D in water at 37° C. IIK12 alone was unable to form hydrogel. However, the addition of IK8D or IK8L promoted gelation (Table 7). SEM images show that IIK12/IK8D gel has a greater number of pores with smaller size than IIK12 gel (FIG. 9). To investigate the effects of IK12 concentration on the strength of the hybrid hydrogels, we performed dynamic frequency sweep on the hybrid hydrogels with varying IIK12 concentrations. It was observed that the stiffness of IIK12/IK8L increased with increasing IIK12 concentration (Table 8). It is found that the stiffness of hybrid hydrogels is comparable to that of peptide hydrogels in the absence of IK8L at lower IIK12 concentrations (Table 5), indicating that the presence of IK8L did not affect gel mechanical strength at lower concentrations. However, the G′ value of IIK12/IK8L is slightly lower than that of IIK12 hydrogel at 1.0% IIK12. Notably, keeping IIK12 constant at a concentration of 1%, gelation was observed for hybrid hydrogels even at 0.8% IK8L. This further demonstrated that the incorporation of IK8L did not affect IIK12 gelation significantly. Similar to IIK12 hydrogel, the IIK12/IK8L hybrid hydrogel exhibited shear-thinning and recovery rheological behaviours (FIG. 10). The viscosity of IIK12/IK8L was reduced rapidly when shear stress was applied, indicating that it is injectable. The rheological behaviour of IIK12/IK8L was further assessed by dynamic step strain amplitude test. The G′ value was −200 Pa for IIK12/IK8L at a strain of 0.5%. When 100% strain was applied to the gel, there was a significant decrease in stiffness. When the strain was reduced to 0.5%, the hybrid hydrogel quickly self-healed and restored its initial stiffness. This property allows the hydrogel to be delivered via syringe while the gel strength is unaffected by the injection process. The reversibility in rheological behaviour is also extremely useful for topical application.

Antimicrobial Activity of IIK12/IK8 Hybrid Hydrogel

The antimicrobial activity of the hybrid hydrogel was investigated against a representative set of clinically relevant microorganisms including Gram-positive S. aureus, Gram-negative E. coli and yeast C. albicans. It has been reported that common bacteria pathogens associated with DFU infection are Gram-positive S. aureus, and Gram-negative E. coli and P. aeruginosa. S. aureus is the predominant pathogen, and E. coli and P. aeruginosa occurred in approximately 10-20% patients[32]. Hybrid hydrogels were prepared with IIK12 at 1%. At the same time, the concentration of IK8L was varied to investigate its effect on the antimicrobial activity of the hybrid hydrogels. Each hydrogel surface was challenged with three pathogens at a cell density of 10⁵ CFU/mL. The microbial proliferation was assessed by optical density (OD) measurement, and viable cells on the hydrogel surface after treatment were quantified by agar plating. As shown in FIG. 11, control gel IIK12 was ineffective in killing the microbes, as demonstrated from OD measurement and agar plating results. The incorporation of antimicrobial peptide, IK8L, rendered antimicrobial activity. Hybrid hydrogels containing 0.128% (1.28 mg/mL) or 0.256% (2.56 mg/mL) IK8L are capable of killing S. aureus, E. coli and C. albicans. Importantly, hybrid hydrogels demonstrated 100% killing upon contact with S. aureus, E. coli and C. albicans. The cationic antimicrobial peptide was released from the hydrogel and was attracted to and interacted with the anionic cell membrane of the microbes via electrostatic interaction. In the presence of microbial cell membrane, IK8L readily folded into secondary β-sheet structures stabilized by the electrostatic interaction, followed by insertion of its hydrophobic residues into the lipid bilayer of the microbes, leading to the physical disruption of microbial cell membrane.

The capability of hybrid hydrogels in killing S. aureus was further investigated by analysing the viable microbes upon contact with IIK12/IK8L at various exposure times. As shown in FIG. 12, S. aureus cells were completely killed after exposure to the hybrid gel surface for half an hour at 2.56 mg/mL of IK8L (i.e. 0.256%), indicating fast killing kinetics. It is noted that the Log (CFU) values at 24 h of incubation were 8.76 and 9.28, for control medium and control gel IIK12, respectively. However, no CFU was observed on the surface of hybrid hydrogels, indicating that 100% of the microbes were killed upon contact with hybrid hydrogels. This finding further proved that IK8L played a critical role in potent antimicrobial action of the hybrid hydrogels.

The antimicrobial activity of peptides and peptide hydrogels in preventing colony formation of the microbes was further studied using a disk diffusion assay technique. Firstly, the sterile filter disc containing IK8L with various concentrations was placed on a freshly S. aureus inoculated agar plate. As shown in FIG. 13A, IK8L effectively inhibited S. aureus colony formation on the agar plates, showing zone of inhibition around the filter disk containing the peptide. Notably, the area of inhibition zone increased with increasing peptide concentration. IIK12/IK8L hybrid hydrogel was then cast on agar plates that had been inoculated with S. aureus, and allowed to incubate for one day at 37° C. Bacterial overgrowth was seen on agar that contact with IIK12 gel and surrounding area, indicating that it is ineffective in preventing S. aureus colony formation (FIG. 13B). In contrast, in the case of IIK12/IK8L, the underlying agar contacting with gel remained clear, demonstrating its ability to prevent S. aureus colony formation (FIG. 13B). The inhibition zone was also observed for the hybrid hydrogel. The area of inhibition zone was larger for IIK12/IK8D hybrid hydrogel due to stronger antimicrobial activity of IK8D than IK8L (FIG. 13C). These findings demonstrate that the antimicrobial peptides can be released out from the hybrid hydrogels.

In Vitro Biocompatibility of IIK12/IK8 Hybrid Hydrogels

Hemolysis is one of the major side effects caused by many cationic peptides and polymers. Hemolytic behaviour of hybrid hydrogels was evaluated after incubation with rat red blood cells. We previously showed that IK8L and IK8D exhibited low hemolytic activity, where HC₁₀ value (the lowest concentration that induced 10% or more hemolysis) was 2000 and 1750 μg/mL, respectively. Similar to the previous findings on IK8L and IK8D, their corresponding hybrid hydrogels demonstrated minimal hemolysis against rat red blood cells even at a concentration of IK8D as high as 10.24 mg/mL (1.024%) (FIG. 14).

In vitro biocompatibility of the peptide gel was further evaluated using human primary dermal fibroblasts (HDFs) and keratinocytes. We first evaluated the cytotoxicity of the peptides IK8L in HDFs. Cell viability was more than 85% up to 500 μg/mL (FIG. 15A). However, cell viability was reduced to 22% when the concentration of IK8L increased to 1000 μg/mL. Notably, HDFs exposed to IIK12/IK8L hydrogel showed cell viability of more than 95% at 1000 μg/mL of IK8L, indicating that the hybrid hydrogel was not cytotoxic at the concentrations tested (FIG. 15B). The in vitro biocompatibility of Mupirocin and polymyxin B, which are commonly used in antimicrobial creams, was compared with the IIK12/IK8L under the same testing conditions (FIGS. 15C and D). Mupirocin exhibited good biocompatibility toward HDFs, as evidenced by more than 80% cell viability even at 2000 μg/mL. Polymyxcin B had a similar cytotoxicity profile in HDFs to IK8L. In the case of keratinocytes, IK8L displayed a dosage dependent cytotoxicity. More than 70% of keratinocytes remained viable when exposed to IK8L at 125 μg/mL (FIG. 16A). IIK12/IK8L showed higher cell viability (close to 100%) than IK8L at 125 μg/mL of IK8L (FIG. 16B). The viability of keratinocytes was 80-90% after it was exposed to mupirocin (FIG. 16C).

Advantageously, the short peptides described herein self-assemble into a hydrogel at lower concentrations than previously prepared hydrogels. Further, the peptide hydrogel is biocompatible and has no cytotoxicity at the concentrations tested and to be used at. The peptide hydrogels may be used for various biomedical applications, for example as a scaffold for 2D and/or 3D cell culture, cell delivery and matrix for controlled release of therapeutic agents.

While the short peptide hydrogels alone have no antimicrobial activity, they may be combined with the previously reported IK8L/IK8D to form a hybrid hydrogel which combines the desirable properties of both peptides. In particular, the biocompatibility, absence of cytotoxicity, and the reversibility of their rheological behaviour (property) of the peptide hydrogels described herein. Thus, these new peptide hydrogels can act as a carrier to deliver a therapeutic agent (e.g. a drug). On the other hand, the IK8L/IK8D peptides contribute antimicrobial activity while forming the hybrid hydrogel at a much lower concentration than previously reported (e.g. 8 (w/v) %), thus mitigating the cytotoxicity problem (e.g. 2 (w/v) %) with using IK8L/IK8D alone.

In particular, the hybrid antimicrobial hydrogel (i.e. IIK12/IK8L) is designed for prevention and treatment of infections, where IIK12 acts as a drug carrier and IK8L acts as an antimicrobial agent. Advantageously, IIK12 forms the hydrogel at lower concentrations. Moreover, it is biocompatible and has no cytotoxicity. Further, the IIK12/IK8L hybrid hydrogel deliver IK8L to the DFU site to eradicate the infections at an effective concentration which kill the microbes while minimising cytotoxicity towards mammalian cells and tissues. Similarly, the other hydrogels described herein are expected to similarly form hybrid hydrogels with similar therapeutic properties while minimising cytotoxicity.

### CONCLUSIONS

In this study, a series of short synthetic peptides was designed with alternating hydrophobic residues (X) and hydrophilic residues (Y). The hydrophobic residues (X) are selected from Ile, Val and Leu, and the hydrophilic residues (Y) are selected from Arg, Lys, Glu and Asp. The peptide amphiphiles self-assemble into hydrogels with tunable mechanical strength, reversible rheological behaviors and sustainable stability. The peptide hydrogels self-assembled from the peptides with 8 amino acids have been proven to be biocompatible with a range of cells including hMSCs, and demonstrated great potential to be used as a cell delivery carrier to support cell proliferation. Peptide amphiphiles comprising 12 amino acids self-assembled into β sheet conformation and formed peptide hydrogels in aqueous solution at low peptide concentrations (0.6% w/v). Beta sheet-forming antimicrobial peptides were incorporated into the hydrogel through electrostatic interaction, to form a hybrid antimicrobial peptide hydrogel. The hybrid hydrogels exhibited shear-thinning and recovery rheological behaviour. The hybrid hydrogels demonstrate broad-spectrum antimicrobial activity against various clinically relevant microbes. Moreover, they demonstrated in vitro biocompatibility. These antimicrobial hybrid peptide hydrogels demonstrate great potential for use in prevention and treatment of bacterial and fungal infections including DFU infection.

### REFERENCES

[1] A. S. Hoffman, Hydrogels for biomedical applications, Advanced Drug Delivery Reviews 54(1) (2002) 3-12.
[2] N. Annabi, A. Tamayol, J. A. Uquillas, M. Akbari, L. E. Bertassoni, C. Cha, G. Camci-Unal, M. R. Dokmeci, N. A. Peppas, A. Khademhosseini, 25th Anniversary Article: Rational Design and Applications of Hydrogels in Regenerative Medicine, Advanced Materials 26(1) (2014) 85-124.
[3] M. C. Cushing, K. S. Anseth, Hydrogel cell cultures, Science 316(5828) (2007) 1133-1134.
[4] S. Baatout, Endothelial differentiation using Matrigel, Anticancer Res. 17(1A) (1997) 451-455.
[5] W. G. Stetlerstevenson, L A. Liotta, D. E. Kleiner, EXTRACELLULAR MATRIX-6—ROLE OF MATRIX METALLOPROTEINASES IN TUMOR INVASION AND METASTASIS, Faseb J. 7(15) (1993) 1434-1441.
[6] B. R. Seo, P. DelNero, C. Fischbach, In vitro models of tumor vessels and matrix: Engineering approaches to investigate transport limitations and drug delivery in cancer, Advanced Drug Delivery Reviews 69 (2014) 205-216.
[7] D. Bosnakovski, M. Mizuno, G. Kim, S. Takagi, M. Okumura, T. Fujinaga, Chondrogenic differentiation of bovine bone marrow mesenchymal stem cells (MSCs) in different hydrogels: Influence of collagen type II extracellular matrix on MSC chondrogenesis, Biotechnology and Bioengineering 93(6) (2006) 1152-1163.
[8] F. Lee, J. E. Chung, M. Kurisawa, An injectable hyaluronic acid-tyramine hydrogel system for protein delivery, Journal of Controlled Release 134(3) (2009) 186-193.
[9] L. S. Wang, J. E. Chung, P. P. Y. Chan, M. Kurisawa, Injectable biodegradable hydrogels with tunable mechanical properties for the stimulation of neurogenesic differentiation of human mesenchymal stem cells in 3D culture, Biomaterials 31(6) (2010) 1148-1157.
[10] H. J. Lee, J. S. Lee, T. Chansakul, C. Yu, J. H. Elisseeff, S. M. Yu, Collagen mimetic peptide-conjugated photopolymerizable PEG hydrogel, Biomaterials 27(30) (2006) 5268-5276.
[11] A. M. Kloxin, A. M. Kasko, C. N. Salinas, K. S. Anseth, Photodegradable Hydrogels for Dynamic Tuning of Physical and Chemical Properties, Science 324(5923) (2009) 59-63.
[12] D. A. Bichara, X. Zhao, H. Bodugoz-Senturk, F. P. Ballyns, E. Oral, M. A. Randolph, L. J. Bonassar, T. J. Gill, O. K. Muratoglu, Porous Poly(Vinyl Alcohol)-Hydrogel Matrix-Engineered Biosynthetic Cartilage, Tissue Engineering Part A 17(3-4) (2011) 301-309.
[13] K. A. Aamer, H. Sardinha, S. R. Bhatia, G. N. Tew, Rheological studies of PLLA-PEO-PLLA triblock copolymer hydrogels, Biomaterials 25(6) (2004) 1087-1093.
[14] A. Basu, K. R. Kunduru, S. Doppalapudi, A. J. Domb, W. Khan, Poly(lactic acid) based hydrogels, Advanced Drug Delivery Reviews 107 (2016) 192-205.
[15] D. Klinger, K. Landfester, Dual Stimuli-Responsive Poly(2-hydroxyethyl methacrylate-co-methacrylic acid) Microgels Based on Photo-Cleavable Cross-Linkers: pH-Dependent Swelling and Light-Induced Degradation, Macromolecules 44(24) (2011) 9758-9772.
[16] S. G. Zhang, T. C. Holmes, C. M. Dipersio, R. O. Hynes, X. Su, A. Rich, SELF-COMPLEMENTARY OLIGOPEPTIDE MATRICES SUPPORT MAMMALIAN-CELL ATTACHMENT, Biomaterials 16(18) (1995) 1385-1393.
[17] S. G. Zhang, T. Holmes, C. Lockshin, A. Rich, SPONTANEOUS ASSEMBLY OF A SELF-COMPLEMENTARY OLIGOPEPTIDE TO FORM A STABLE MACROSCOPIC MEMBRANE, Proceedings of the National Academy of Sciences of the United States of America 90(8) (1993) 3334-3338.
[18] M. E. Davis, P. C. H. Hsieh, T. Takahashi, Q. Song, S. G. Zhang, R. D. Kamm, A. J. Grodzinsky, P. Anversa, R. T. Lee, Local myocardial insulin-like growth factor 1 (IGF-1) delivery with biotinylated peptide nanofibers improves cell therapy for myocardial infarction, Proceedings of the National Academy of Sciences of the United States of America 103(21) (2006) 8155-8160.
[19] F. Gelain, D. Bottai, A. Vescovi, S. G. Zhang, Designer Self-Assembling Peptide Nanofiber Scaffolds for Adult Mouse Neural Stem Cell 3-Dimensional Cultures, Plos One 1(2) (2006).
[20] Y. Chau, Y. Luo, A. C. Y. Cheung, Y. Nagai, S. G. Zhang, J. B. Kobler, S. M. Zeitels, R. Langer, Incorporation of a matrix metalloproteinase-sensitive substrate into self-assembling peptides—A model for biofunctional scaffolds, Biomaterials 29(11) (2008) 1713-1719.
[21] T. C. Holmes, S. de Lacalle, X. Su, G. S. Liu, A. Rich, S. G. Zhang, Extensive neurite outgrowth and active synapse formation on self-assembling peptide scaffolds, Proceedings of the National Academy of Sciences of the United States of America 97(12) (2000) 6728-6733.
[22] F. Gelain, L. D. Unsworth, S. G. Zhang, Slow and sustained release of active cytokines from self-assembling peptide scaffolds, Journal of Controlled Release 145(3) (2010) 231-239.
[23] B. Ozbas, J. Kretsinger, K. Rajagopal, J. P. Schneider, D. J. Pochan, Salt-triggered peptide folding and consequent self-assembly into hydrogels with tunable modulus, Macromolecules 37(19) (2004) 7331-7337.
[24] D. A. Salick, D. J. Pochan, J. P. Schneider, Design of an Injectable beta-Hairpin Peptide Hydrogel That Kills Methicillin-Resistant Staphylococcus aureus, Advanced Materials 21(41) (2009) 4120-+.
[25] D. A. Salick, J. K. Kretsinger, D. J. Pochan, J. P. Schneider, Inherent antibacterial activity of a peptide-based beta-hairpin hydrogel, J. Am. Chem. Soc. 129(47) (2007) 14793-14799.
[26] L Haines-Butterick, K. Rajagopal, M. Branco, D. Salick, R. Rughani, M. Pilarz, M. S. Lamm, D. J. Pochan, J. P. Schneider, Controlling hydrogelation kinetics by peptide design for three-dimensional encapsulation and injectable delivery of cells, Proceedings of the National Academy of Sciences of the United States of America 104(19) (2007) 7791-7796.
[27] C. A. E. Hauser, R. S. Deng, A. Mishra, Y. H. Loo, U. Khoe, F. R. Zhuang, D. W. Cheong, A. Accardo, M. B. Sullivan, C. Riekel, J. Y. Ying, U. A. Hauser, Natural tri- to hexapeptides self-assemble in water to amyloid beta-type fiber aggregates by unexpected alpha-helical intermediate structures, Proceedings of the National Academy of Sciences of the United States of America 108(4) (2011) 1361-1366.
[28] A. Mishra, Y. H. Loo, R. H. Deng, Y. J. Chuah, H. T. Hee, J. Y. Ying, C. A. E. Hauser, Ultrasmall natural peptides self-assemble to strong temperature-resistant helical fibers in scaffolds suitable for tissue engineering, Nano Today 6(3) (2011) 232-239.
[29] Y. Loo, Y. C. Wong, E. Z. Cai, C. H. Ang, A. Raju, A. Lakshmanan, A. G. Koh, H. J. Zhou, T. C. Lim, S. M. Moochhala, C. A. E. Hauser, Ultrashort peptide nanofibrous hydrogels for the acceleration of healing of burn wounds, Biomaterials 35(17) (2014) 4805-4814.
[30] W. Y. Seow, G. Salgado, E. B. Lane, C. A. E. Hauser, Transparent crosslinked ultrashort peptide hydrogel dressing with high shape-fidelity accelerates healing of full-thickness excision wounds, Scientific Reports 6 (2016).
[31] L. I. F. Moura, A. M. A. Dias, E. Carvalho, H. C. de Sousa, Recent advances on the development of wound dressings for diabetic foot ulcer treatment-A review, Acta Biomaterialia 9(7) (2013) 7093-7114.
[32] F. W. Gemechu, F. Seemant, C. A. Curley, Diabetic Foot Infections, American Family Physician 88(3) (2013) 177-184.
[33] P. Davies, S. McCarty, K. Hamberg, Silver-containing foam dressings with Safetac: a review of the scientific and clinical data, Journal of Wound Care 26(6) (2017) S1-S30.
[34] H. K. R. Nair, Nano-colloidal silver and chitosan bioactive wound dressings in managing diabetic foot ulcers: case series, Journal of Wound Care 27(9) (2018) S32-S36.
[35] S. G. Zhang, Emerging biological materials through molecular self-assembly, Biotechnology Advances 20(5-6) (2002) 321-339.
[36] I. M. Geisler, J. P. Schneider, Evolution-Based Design of an Injectable Hydrogel, Advanced Functional Materials 22(3) (2012) 529-537.
[37] Z. Y. Ong, J. C. Cheng, Y. Huang, K. J. Xu, Z. K. Ji, W. M. Fan, Y. Y. Yang, Effect of stereochemistry, chain length and sequence pattern on antimicrobial properties of short synthetic beta-sheet forming peptide amphiphiles, Biomaterials 35(4) (2014) 1315-1325.
[38] Z. Y. Ong, SJ. Gao, Y. Y. Yang, Short Synthetic beta-Sheet Forming Peptide Amphiphiles as Broad Spectrum Antimicrobials with Antibiofilm and Endotoxin Neutralizing Capabilities, Advanced Functional Materials 23(29) (2013) 3682-3692.
[39] H. Wu, Z. Y. Ong, S. Q. Liu, Y. Li, N. Wiradharma, Y. Y. Yang, J. Y. Ying, Synthetic beta-sheet forming peptide amphiphiles for treatment of fungal keratitis, Biomaterials 43 (2015) 44-49.

### Tables

TABLE 1
Peptide design and molecular
weight characterization.

| | | | Theo- | Meas- | SEQ |
|---|---|---|---|---|---|
| | Peptide | Peptide | retical | ured | ID |
| Entry | sequences | code | M_{w} | M_{w} | No. |
| 1 | IRVE | | 514.63 | | 6 |
| 2 | IRVEIK | | 755.96 | | 7 |
| 3 | IRVEIEVK | IRK8 | 984.21 | 984.3 | 8 |
| 4 | IRVEIKVE | IRE8 | 984.21 | 984.5 | 9 |
| 6 | IEVEIRVK | IVK8 | 984.21 | 984.6 | 10 |
| 7 | IEIEIRIK | IIK8 | 1012.27 | 1012.7 | 11 |
| 8 | IELEIRLK | ILK8 | 1012.27 | 1013.6 | 12 |
| 9 | IRVEIRVE | IRV12 | 1509.83 | 1510.4 | 13 |
| | IRVE | | | | |
| 10 | IEVEIEVK | IEV12 | 1453.79 | 1455.2 | 14 |
| | IRVK | | | | |
| 11 | IRVKIEVE | IVK12 | 1480.86 | 1481.3 | 15 |
| | IRVK | | | | |
| 12 | IRIKIEIE | IIK12 | 1522.95 | 1524.4 | 16 |
| | IRIK | | | | |
| 13 | EVKIEVRI | Reverse | | | 19 |
| | | IRE8 | | | |
| 14 | KVRIEVEI | Reverse | | | 20 |
| | | IVK8 | | | |
| 15 | KIRIEIEI | Reverse | | | 21 |
| | | IIK8 | | | |
| 16 | KLRIELEI | Reverse | | | 22 |
| | | ILK8 | | | |
| 17 | EVRIEVRI | Reverse | | | 23 |
| | EVRI | IRV12 | | | |
| 18 | KVRIKVEI | Reverse | | | 24 |
| | EVEI | IEV12 | | | |
| 19 | KVRIEVEI | Reverse | | | 25 |
| | KVRI | IVK12 | | | |
| 20 | KIRIEIEI | Reverse | | | 26 |
| | KIRI | IIK12 | | | |

The peptides of SEQ ID No. 6 to 16 were amidated at the C terminus. The peptides of SEQ ID No. 19 to 26 were amidated at the C-terminus and N-acetylated at the N-terminus.

TABLE 2
Effects of varying charge distribution and
hydrophobicity on gelation properties.

| | | | | Gelation | |
|---|---|---|---|---|---|
| | | | Gelation | in | |
| | | | in DI | DI water | |
| | | | water | at | Stabil- |
| | | Peptide | at R.T. | 37° C. | ity |
| | Peptide | conc. | (via | (via | in |
| Entry | sequences | (%) | tilting) | tilting) | PBS |
| 1 | IRVE | 10 | No | | |
| 2 | IRVEIK | 1.0 | No | | |
| | | 2.0 | No | | |
| 3 | IRVEIEVK | 1.0 | No | | |
| | | 2.0 | No | | |
| 4 | IRVEIKVE | 1.0 | No | | |
| | | 2.0 | 3 days, | 1 days | |
| | | | clear | | |
| 6 | IEVEIRVK | 1.0 | 3 days, | 2 days | Yes |
| | | | clear | | |
| | | 1.5 | 1 day, | overnight | Yes |
| | | | clear | | |
| | | 2.0 | 60 min, | 30 min | Yes |
| | | | clear | | |
| 7 | IEIEIRIK | 1.0 | 1 day, | overnight | Yes |
| | | | clear | | |
| | | 1.5 | 30 min | 20 min | Yes |
| | | 2.0 | 30 min | 20 min | Yes |
| 8 | IELEIRLK | 1.0 | 1 day | overnight | Yes |
| | | 1.5 | 30 min | 15 min | Yes |
| | | 2.0 | 20 min | 10 min | Yes |
| 9 | IRVEIRVE | 0.5 | No | | |
| | IRVE | | | | |
| | | 1.0, | | Gel, | |
| | | 2.0 | | slightly | |
| | | | | cloudy | |
| 10 | IEVEIEVK | 0.5 | No | | |
| | IRVK | | | | |
| | | 1.0, | | Gel | |
| | | 2.0 | | | |
| 11 | IRVKIEVE | 0.6, | | Gel | |
| | IRVK | 1.0 | | | |
| 12 | IRIKIEIE | 0.6, | | Gel | |
| | IRIK | 1.0 | | | |

TABLE 3
G′ (storage modulus) and G″ (loss modulus) values of peptide hydrogels self-assembled from
IVK8, IIK8 and ILK8 at frequency of 1 Hz.

| | Peptide conc. | Gelation | | G’ | |
|---|---|---|---|---|---|
| Peptides | (wt %) | temp. (° C.) | Medium | (Pa) | G”(Pa) |
| IVK8 | 1.0 | R.T. | H₂O | 23 | 3 |
| | 1.5 | R.T. | H₂O | 84 | 8 |
| | 1.75 | R.T. | H₂O | 282 | 41 |
| | 1.5 | 37 | H₂O | 489 | 15 |
| | 1.5 | 37 | PBS | 892 | 90 |
| | 2.5 | 37 | PBS | 10108 | 2253 |
| | 1.5 | 37 | DMEM | 2353 | 904 |
| | 2.0 | 37 | DMEM | 5347 | 1555 |
| IIK8 | 1.5 | R.T. | H₂O | 125 | 22 |
| | 1.5 | 37 | H₂O | 707 | 32 |
| | 2.0 | 37 | H₂O | 1139 | 90 |
| | 1.5 | 37 | DMEM | 1206 | 172 |
| ILK8 | 1.5 | R.T. | H₂O | 332 | 40 |

TABLE 4
Stability of peptide hydrogels self-assembled from IVK8 in the presence of DMEM. The hydrogels were formed
at 37° C.. For the entry with initial peptide
concentration 1.0 (w/v) %, the final peptide concentration: 0.8%, 0.7%, 0.6% and 0.5% for
ratios of 80:20, 70:30, 60:40 and 50:50, respectively.
The same applies to the other initial concentrations.

| Peptide | | | | | |
|---|---|---|---|---|---|
| conc. | | Ratios (peptide: DMEM) | | | |
| (w/v) % | Days | 80:20 | 70:30 | 60:40 | 50:50 |
| 1.0 | 1 | Y | Y | Y | Y |
| | 7 | Y | Y | Y | N |
| | 15 | N | N | N | N |
| 1.2 | 1 | Y | Y | Y | Y |
| | 7 | Y | Y | Y | N |
| | 15 | N | Y | N | N |
| 1.5 | 1 | Y | Y | Y | Y |
| | 7 | Y | Y | Y | N |
| | 15 | Y | Y | Y | N |

TABLE 5
G′ (storage modulus) and G″ (loss modulus) values of peptide
hydrogels self-assembled from IIK12 at frequency of 1 Hz.

| | Peptide conc. | | | |
|---|---|---|---|---|
| Peptides | (wt %) | Medium | G’ (Pa) | G”(Pa) |
| IIK12 | 0.6 | H₂O | 65 | 7 |
| | 0.8 | H₂O | 131 | 24 |
| | 1.0 | H₂O | 279 | 48 |
| | 1.5 | H₂O | 598 | 66 |
| | 2.0 | H₂O | 865 | 69 |
| | 1.0 | DMEM | 1206 | 176 |

The hydrogels were incubated at 37° C. overnight.

TABLE 6
Gelation of antimicrobial peptide hybrid hydrogels in water.

| | IIK12 | | Molar ratio | Gelation |
|---|---|---|---|---|
| | conc. | Molar ratio of | of IIK12 | at 37° C. |
| Peptides | (wt %) | IIK12 to IK8D | to IK8L | for overnight |
| IIK12 | 0.5 | 0 | 0 | N |
| IIK12/IK8D | 0.5 | 0.5 | | Y |
| IIK12/IK8L | 0.5 | | 0.5 | Y |

TABLE 7
Gelation of antimicrobial peptide hybrid hydrogels.

| | IIK12 or | IK8L | | Gelation at room |
|---|---|---|---|---|
| | IVK12 conc. | conc. | | temperature (via |
| Peptides | (wt %) | (wt %) | Medium | tilting) |
| IIK12/IK8L | 1.0 | 0.8 | H₂O | 60 min |
| | 1.0 | 0.8 | PBS | 5 min |
| | 1.0 | 0.8 | NaOAC | 5 min |
| | 1.0 | 0.8 | NaCl | 5 min |
| IVK12/IK8L | 1.0 | 0.8 | H₂O | 60 min |
| | 1.0 | 0.8 | PBS | 10 min |
| | 1.0 | 0.8 | NaOAC | 10 min |
| | 1.0 | 0.8 | NaCl | 10 min |

Final salt concentration in the hydrogel: 150 mM

TABLE 8
G′ (storage modulus) and G″ (loss modulus) values of
antimicrobial peptide hydrogels at frequency of 1 Hz.

| | IIK12conc. | IK8L conc. | | | |
|---|---|---|---|---|---|
| Peptides | (wt %) | (wt %) | Medium | G’ (Pa) | G”(Pa) |
| IIK12/IK8L | 0.6 | 0.8 | H₂O | 60 | 23 |
| | 0.8 | 0.8 | H₂O | 123 | 26 |
| | 1.0 | 0.8 | H₂O | 188 | 23 |

The hydrogels were incubated at 37° C. overnight.

1. A peptide comprising an amino acid sequence of alternating hydrophobic amino acids (X) and hydrophilic amino acids (Y), wherein each hydrophobic amino acid is independently selected from isoleucine (I), valine (V) and leucine (L), each hydrophilic amino acid is independently selected from arginine (R), lysine (K), glutamic acid (E), and aspartic acid (D), at least one hydrophilic amino acid is selected from arginine and lysine, at least one hydrophilic amino acid is selected from glutamic acid and aspartic acid, and the amino acid sequence contains at least 8 amino acids, wherein there is at least one arginine and at least one lysine in the amino acid sequence and the hydrophilic amino acids are selected such that the peptide has a net neutral charge or net positive charge of +2, and wherein the amino acid sequence is not IRVEIEVK (SEQ ID No. 8).

2. (canceled)

3. The peptide according to claim 1, wherein the amino acid sequence has an even number of amino acids.

4. The peptide according to claim 1, wherein the amino acid sequence has 8 or 12 amino acids.

5. (canceled)

6. The peptide according to claim 1, wherein four sequential hydrophilic amino acids (Y₁, Y₂, Y₃ and Y₄) in the amino acid sequence of alternating hydrophobic amino acids (X) and hydrophilic amino acids (Y) are selected such that Y₁ and Y₂ are each independently selected from glutamic acid and aspartic acid, and Y₃ and Y₄ are each independently selected from arginine and lysine.

7. The peptide according to claim 1, wherein four sequential hydrophilic amino acids (Y₁, Y₂, Y₃ and Y₄) in the amino acid sequence of alternating hydrophobic amino acids (X) and hydrophilic amino acids (Y) are selected such that Y₁ and Y₃ are each independently selected from arginine and lysine, and Y₂ and Y₄ are each independently selected from glutamic acid and aspartic acid.

8. The peptide according to claim 1, wherein six sequential hydrophilic amino acids (Y₁, Y₂, Y₃, Y₄, Y₅, and Y₆) in the amino acid sequence of alternating hydrophobic amino acids (X) and hydrophilic amino acids (Y) are selected such that Y₁, Y₃, and Y₅ are each independently selected from arginine and lysine, and Y₂, Y₄, and Y₆ are each independently selected from glutamic acid and aspartic acid.

9. The peptide according to claim 1, wherein six sequential hydrophilic amino acids (Y₁, Y₂, Y₃, Y₄, Y₅, and Y₆) in the amino acid sequence of alternating hydrophobic amino acids (X) and hydrophilic amino acids (Y) are selected such that Y₁, Y₂, and Y₃ are each independently selected from glutamic acid and aspartic acid, and Y₄, Y₅, and Y₆ are each independently selected from arginine and lysine.

10. The peptide according to claim 1, wherein six sequential hydrophilic amino acids (Y₁, Y₂, Y₃, Y₄, Y₅, and Y₆) in the amino acid sequence of alternating hydrophobic amino acids (X) and hydrophilic amino acids (Y) are selected such that Y₁, Y₂, Y₅, and Y₆ are each independently selected from arginine and lysine, and Y₃, and Y₄ are each independently selected from glutamic acid and aspartic acid.

11. (canceled)

12. The peptide according to claim 1, wherein at least half of the hydrophobic amino acids in the amino acid sequence are isoleucine or leucine or wherein there are 12 amino acids in the amino acid sequence and the hydrophobic amino acids are each independently selected from isoleucine and valine.

13. (canceled)

14. The peptide according to claim 1, wherein the amino acid sequence is any one of the following: SEQ ID No. 9, SEQ ID No. 10, SEQ ID No. 11, SEQ ID No. 12, SEQ ID No. 14, SEQ ID No. 15, SEQ ID No. 16, SEQ ID No. 19, SEQ ID No. 20, SEQ ID No. 21, SEQ ID No. 22, SEQ ID No. 24, SEQ ID No. 25, and SEQ ID No. 26.

15. (canceled)

16. A composition comprising a hydrogel formed of a plurality of peptides according to claim 1 in a beta-sheet conformation and water or a dried form of the hydrogel.

17. The composition according to claim 16, wherein a concentration of the plurality of peptides is at least 0.6% w/v.

18. The composition according to claim 17, wherein the concentration is at most 5% w/v.

19. (canceled)

20. The composition according to claim 16, comprising one or more salt selected from PBS, DMEM, MEM, a potassium salt, and a sodium salt.

21. The composition according to claim 16, wherein the peptide has the amino sequence of SEQ ID No. 12, SEQ ID No. 15, SEQ ID No. 16, SEQ ID No. 22, SEQ ID No. 25, or SEQ ID No. 26.

22. The composition according to claim 16, comprising a therapeutic agent.

23. The composition according to claim 16, comprising a second peptide of SEQ ID No. 17 or SEQ ID No. 18.

24. (canceled)

25. (canceled)

26. A method of treating a bacterial and/or a fungal infection, the method comprising administering a therapeutically effective amount of the composition according to claim 23 to a subject suffering from a bacterial and/or a fungal infection.

27. A method to form a hydrogel, the method comprising mixing a peptide according to claim 1 in water to form a hydrogel; and isolating the hydrogel.

28. The method according to claim 27, wherein the mixing step is done in the presence of a salt, or wherein the mixing step is done at a temperature of 20° C. to 40° C., or the method further comprising drying the hydrogel to form a dried hydrogel suitable for reconstitution to the hydrogel.

29. (canceled)

30. (canceled)

31. An in-vitro method of growing cells, the method comprises providing a mixture of the peptide according to claim 1, cell culture medium, and a population of cells to form the gel containing the population of cells; and incubating the gel under suitable conditions to grow the population of cells, wherein the cell is any one selected from a healthy cell, stem cell which is used for therapy or to grow tissues, and a cancer cell which is used to grow a tumor for in vitro and in vivo studies.

32. (canceled)


SEQUENCE LISTING (26 sequences)

SEQ ID NO: 1 - protein, 16 aa, Saccharomyces cerevisiae
AEAEAKAKAEAEAKAK

SEQ ID NO: 2 - protein, 16 aa, Artificial Sequence
  N-acetylated, carboxyamide
  MOD_RES (1)..(1): ACETYLATION
  MOD_RES (16)..(16): AMIDATION
RADARADARADARADA

SEQ ID NO: 3 - protein, 16 aa, Artificial Sequence
  N-acetylated, carboxyamide
  MOD_RES (1)..(1): ACETYLATION
  MOD_RES (16)..(16): AMIDATION
RARADADARARADADA

SEQ ID NO: 4 - protein, 20 aa, Artificial Sequence
  a central tetrapeptide type II′ beta-turn flanked by alternating valine (hydrophobic) and lysine (hydrophilic) residues
  SITE (9)..(9): Xaa (X)  is D-valine
  TURN (9)..(12): Type II′ beta-turn
VKVKVKVKXPPTVKVKVKVK

SEQ ID NO: 5 - protein, 6 aa, Artificial Sequence
  N-acetylated, carboxyamide
  MOD_RES (1)..(1): ACETYLATION
  MOD_RES (6)..(6): AMIDATION
ILVAGK

SEQ ID NO: 6 - protein, 4 aa, Artificial Sequence
  4 amino acid peptide
  MOD_RES (4)..(4): AMIDATION
IRVE

SEQ ID NO: 7 - protein, 6 aa, Artificial Sequence
  6 amino acid peptide
  MOD_RES (6)..(6): AMIDATION
IRVEIK

SEQ ID NO: 8 - protein, 8 aa, Artificial Sequence
  IRK8
  MOD_RES (8)..(8): AMIDATION
IRVEIEVK

SEQ ID NO: 9 - protein, 8 aa, Artificial Sequence
  IRE8
  MOD_RES (8)..(8): AMIDATION
IRVEIKVE

SEQ ID NO: 10 - protein, 8 aa, Artificial Sequence
  IVK8
  MOD_RES (8)..(8): AMIDATION
IEVEIRVK

SEQ ID NO: 11 - protein, 8 aa, Artificial Sequence
  IIK8
  MOD_RES (8)..(8): AMIDATION
IEIEIRIK

SEQ ID NO: 12 - protein, 8 aa, Artificial Sequence
  ILK8
  MOD_RES (8)..(8): AMIDATION
IELEIRLK

SEQ ID NO: 13 - protein, 12 aa, Artificial Sequence
  IRV12
  MOD_RES (12)..(12): AMIDATION
IRVEIRVEIRVE

SEQ ID NO: 14 - protein, 12 aa, Artificial Sequence
  IEV12
  MOD_RES (12)..(12): AMIDATION
IEVEIEVKIRVK

SEQ ID NO: 15 - protein, 12 aa, Artificial Sequence
  IVK12
  MOD_RES (12)..(12): AMIDATION
IRVKIEVEIRVK

SEQ ID NO: 16 - protein, 12 aa, Artificial Sequence
  IIK12
  MOD_RES (12)..(12): AMIDATION
IRIKIEIEIRIK

SEQ ID NO: 17 - protein, 8 aa, Artificial Sequence
  IK8L
IRIKIRIK

SEQ ID NO: 18 - protein, 8 aa, Artificial Sequence
  IK8D, all D-amino acids
  SITE (1)..(8): All D-amino acids
IRIKIRIK

SEQ ID NO: 19 - protein, 8 aa, Artificial Sequence
  Reverse IRE8
  MOD_RES (1)..(1): ACETYLATION
  MOD_RES (8)..(8): AMIDATION
EVKIEVRI

SEQ ID NO: 20 - protein, 8 aa, Artificial Sequence
  Reverse IVK8
  MOD_RES (1)..(1): ACETYLATION
  MOD_RES (8)..(8): AMIDATION
KVRIEVEI

SEQ ID NO: 21 - protein, 8 aa, Artificial Sequence
  Reverse IIK8
  MOD_RES (1)..(1): ACETYLATION
  MOD_RES (8)..(8): AMIDATION
KIRIEIEI

SEQ ID NO: 22 - protein, 8 aa, Artificial Sequence
  Reverse ILK8
  MOD_RES (1)..(1): ACETYLATION
  MOD_RES (8)..(8): AMIDATION
KLRIELEI

SEQ ID NO: 23 - protein, 12 aa, Artificial Sequence
  Reverse IRV12
  MOD_RES (1)..(1): ACETYLATION
  MOD_RES (12)..(12): AMIDATION
EVRIEVRIEVRI

SEQ ID NO: 24 - protein, 12 aa, Artificial Sequence
  Reverse IEV12
  MOD_RES (1)..(1): ACETYLATION
  MOD_RES (12)..(12): AMIDATION
KVRIKVEIEVEI

SEQ ID NO: 25 - protein, 12 aa, Artificial Sequence
  Reverse IVK12
  MOD_RES (1)..(1): ACETYLATION
  MOD_RES (12)..(12): AMIDATION
KVRIEVEIKVRI

SEQ ID NO: 26 - protein, 12 aa, Artificial Sequence
  Reverse IIK12
  MOD_RES (1)..(1): ACETYLATION
  MOD_RES (12)..(12): AMIDATION
KIRIEIEIKIRI